(19) United States
(12) Patent Application Publication
(10) Pub. No.: US 2025/0049156 A1
(43) Pub. Date: Feb. 13, 2025

(54) GLOVE HAVING DISCREET GRIPPING ZONES IN ONE OF THE PALM REGION AND WRIST REGION

(71) Applicant: Summit Glove Inc., Minerva, OH (US)

(72) Inventor: James L. Hull, Malvern, OH (US)

(73) Assignee: Summit Glove Inc., Minerva, OH (US)

(21) Appl. No.: 18/366,475

(22) Filed: Aug. 7, 2023

(51) Int. Cl. A41D 19/00

(52) U.S. Cl. CPC A41D 19/0062; A41D 19/0003; A41D 19/0082; A41D 2400/80; A61B 42/10

(57) ABSTRACT

A glove including a wrist region, a palm region, and a plurality of digit regions. A gripping zone is provided on one or both of the palm region and wrist region. The gripping zone includes concave indentations formed in the glove material which project into the glove's interior cavity. The glove's circumference extending through the gripping zone is reduced. When performing a task while wearing the glove, frictional contact between the gripping zone and the person's skin tends to resist movement of the palm or wrist region relative to the person's hand. This helps to keep the digit regions of the glove in sufficient contact with the user's fingers and thumb to maintain tactile sensitivity and dexterity, even when the glove is worn for an extended period of time.

### TECHNICAL FIELD

This disclosure is directed to gloves. More particularly, the disclosure relates to gloves for use in tasks which require dexterity and tactile sensitivity. Specifically, the present disclosure relates to a glove having at least one gripping zone in a palm region of the glove. Each gripping zone locally reduces the circumference of the palm region such that there is direct contact between the gripping zone and the palm or wrist. When the glove is used, the frictional contact between the gripping zone and the user's skin helps to resist movement of the palm region and thereby of the digit regions of the glove relative to the user's palm, wrist and thereby their fingers.

### BACKGROUND ART

Nitrile or nitrile rubber gloves are well known for use in the medical field. One of the problems with previously known medical gloves is that tactile sensitivity and dexterity may be reduced or lost because of the presence of glove material between the user's sensitive fingertips and the patient or equipment contacted with the user's gloved hand. If surgical gloves are worn for a long period of time there is a tendency for the glove to stretch. The stretching can result in the gloves losing their ability to cling to the user's hand and, more particularly, to the user's fingertips. When the glove material stretches and stops clinging tightly to the user thumb or fingers, the glove material can tend to slide along the length of the thumb or fingers. There is therefore a tendency for the user's tactile sensitivity and finger dexterity to deteriorate over the course of a few hours while wearing the gloves.

The reduction or loss of tactile sensitivity and dexterity may be exacerbated by a reduction in the ability of the glove to closely adhere to the user's palm or wrist over time.

### SUMMARY OF THE INVENTION

An improved glove is disclosed herein. The glove has one or more gripping zones provided in the palm region of the glove. These one or more gripping zones tend to enable the glove to continue to grip onto the user's palm and wrist region over time, thereby aiding the glove to remain in clinging-engagement with the user's hand during use of the glove. Tactile sensitivity and finger dexterity while wearing the glove is therefore maintained for a longer period of time relative to previously-known gloves.

The glove according to the present disclosure includes a wrist region, a palm region extending outwardly from the wrist region, and a plurality of digit regions extending outwardly from the palm region. Each digit region has a longitudinal axis extending from a tip of the digit region to the palm region of the glove. An interior cavity is bounded and defined by the wrist region, the palm region, and the plurality of digit regions. The interior cavity is accessible via an opening defined by the wrist region.

In one aspect, an exemplary embodiment of the present disclosure may provide a glove comprising a wrist region; a palm region extending outwardly from the wrist region; a plurality of digit regions extending outwardly from the palm region; and at least one gripping zone provided on at least one of the palm region and the wrist region of the glove. The gripping zone includes one or more concave indentations in the material of the glove. The concave indentation forms a projection which extends into a part of the interior cavity of the glove as defined by the palm region or wrist region. The projection contacts the user's palm, back of the hand or wrist received within the part of the interior cavity and frictionally engages the same This frictional engagement opposes the tendency of the person's hand or wrist to slide relative to the person's finger or thumb during performance of a task while wearing the glove.

In one embodiment, the at least one gripping zone may include one or more concave indentations formed in a material of the glove, wherein the one or more concave indentations project into an interior cavity of the glove. In one embodiment, the at least one gripping zone may further include one or more convex projections alternating with the one or more concave indentations, wherein the one or more convex projections extend outwardly from an exterior surface of the glove. In one embodiment, the alternating concave indentations and convex projections may form a wave structure having a width and a height. In one embodiment, the at least one gripping zone may be oriented orthogonally to an imaginary longitudinal axis of the glove, wherein the imaginary longitudinal axis extends from a tip of one of the plurality of digit regions to the palm region of the glove.

In one embodiment, the plurality of digit regions may include a thumb region, and the at least one gripping zone is provided on the palm region between the thumb region and a bottom end of the wrist region. In one embodiment, the at least one gripping zone may be generally U-shaped in cross-section when looking in a direction towards a tip of the thumb region. In one embodiment, the at least one gripping zone may be formed partially in a front of the palm region, partially in a first side of the palm region, and partially in a back of the palm region.

In one embodiment, the plurality of digit regions may include a little finger region, and the at least one gripping zone is provided on the palm region between the little finger region, and a bottom end of the wrist region. In one embodiment, the at least one gripping zone may be generally U-shaped in cross-section when looking in a direction towards a tip of the little finger region. In one embodiment, the at least one gripping zone may be formed partially in a front of the palm region, partially in a second side of the palm region, and partially in a back of the palm region.

In one embodiment, the plurality of digit regions may include a thumb region and a little finger region, and wherein the at least one gripping zone may comprise a first gripping zone and a second gripping zone, wherein the first gripping zone is provided on the palm region between the thumb region and a bottom end of the wrist region; and the second gripping zone is provided on the palm region between the little finger region and the bottom end of the wrist region. In one embodiment, the first gripping zone and the second gripping zone may be opposed and laterally aligned with one another.

In one embodiment, the at least one gripping zone may comprise one or more gripping zones provided in the wrist region. In one embodiment, the one or more gripping zones may include a gripping zone provided in one or both of a front and a back of the wrist region. In one embodiment, the one or more gripping zones comprises gripping zones in each of the front and back of the wrist region, and wherein the gripping zone in the front is laterally spaced from and laterally aligned with the gripping zone in the back. In one embodiment, the at least one gripping zone may further comprise a gripping zone provided in the palm region, and the gripping zone in the palm region is laterally spaced from the gripping zone provided in each of the front and the back of the glove. In one embodiment, the at least one gripping zone in the palm region may be longitudinally offset relative to the gripping zone provided in at least one of the front and the back of the glove.

In one embodiment, the glove may further include texturing of an exterior surface of at least a portion of the at least one gripping zone. In one embodiment, the texturing may be applied through micro-etching. In one embodiment, the glove is an ambidextrous glove. In one embodiment, the glove is a hand-specific glove.

In one embodiment, a digit region of the plurality of digit regions includes a fingertip region and a remaining portion, wherein the fingertip region originates at a tip of the digit region and extends towards the palm region, wherein the fingertip region terminates in a location which will be adjacent a first knuckle of a person's finger or thumb when the glove is worn; wherein the remaining portion extends from the fingertip region to the palm region; and wherein the fingertip region is of a reduced circumference relative to the remaining portion. In one embodiment, the glove may further comprise a transition portion provided on the remaining portion, wherein the transition portion extends outwardly from the fingertip region, and wherein the transition portion tapers in circumference moving in a direction from the palm region towards the fingertip region. In one embodiment, the fingertip region may be of a bullet-tip configuration.

In one aspect, an exemplary embodiment of the present disclosure may provide a method comprising inserting a person's hand through an opening defined in a wrist region of a glove; receiving each finger or thumb of the person's hand into a respective one of a plurality of digit regions of the glove; receiving a palm and back of the person's hand into a palm region of the glove, wherein the palm region extends outwardly from the plurality of digit regions; receiving the person's wrist into the wrist region of the glove; providing at least one gripping zone in at least one of the wrist region and the palm region of the glove; and frictionally retaining the one of the palm region and the wrist region of the glove in position on the person's palm and back of the hand, or on the person's wrist with the at least one gripping zone.

In one embodiment, the method may further comprise providing the at least one gripping zone as one or more stays formed in a material of the glove, wherein each of the one or more stays is formed as an arcuate concave indentation which extends into an interior cavity of the glove. In one embodiment, the method may further comprise applying texturing on an exterior surface of the at least one gripping zone. In one embodiment, the texturing may be applied to the exterior surface of the one or more stays via a micro-etching process.

In one aspect, an exemplary embodiment of the present disclosure may provide a glove comprising a wrist region; a palm region extending outwardly from the wrist region; a plurality of digit regions extending outwardly from the palm region; at least one gripping zone provided on at least one of the palm region and the wrist region of the glove; and wherein a circumference of the glove passing through the at least one gripping zone is reduced relative to portions of the glove located longitudinally immediately adjacent the at least one gripping zone.

In one aspect, an exemplary embodiment of the present disclosure may provide a method comprising inserting a person's hand through an opening defined in a wrist region of a glove; receiving each finger or thumb of the person's hand into a respective one of a plurality of digit regions of the glove; receiving a palm and back of the person's hand into a palm region of the glove, wherein the palm region extends outwardly from the plurality of digit regions; receiving the person's wrist into the wrist region of the glove; providing at least one gripping zone in at least one of the wrist region and the palm region of the glove; reducing a circumference of the at least one of the wrist region and the palm region of the glove with the at least one gripping zone; and frictionally retaining the one of the palm region and the wrist region of the glove in position on the person's palm and back of the hand, or on the person's wrist with the at least one gripping zone.

### BRIEF DESCRIPTION OF THE DRAWINGS

Sample embodiments of the present disclosure are set forth in the following description, are shown in the drawings and are particularly and distinctly pointed out and set forth in the appended claims.

FIG. 1 is a front elevation view of a first example of a first embodiment of a glove in accordance with the present disclosure, wherein the first example is an ambidextrous glove shown worn on a left hand of a user, showing discrete gripping zones formed in a palm region and a wrist region of the glove, and showing fingertip regions of reduced circumference;

FIG. 2 is a rear elevation view of the glove illustrated in FIG. 1;

FIG. 3 is a first side elevation view of the glove of FIG. 1;

FIG. 4 is a second side elevation view of the glove of FIG. 1;

FIG. 5A is a front elevation view of the highlighted area of the glove shown in FIG. 1;

FIG. 5B is a first side elevation view of the highlighted area taken along line 5B-5B of FIG. 5A;

FIG. 6A is an enlarged longitudinal cross-section of the highlighted are taken along line 6A-6A of FIG. 5B;

FIG. 6B is an enlarged longitudinal cross-section of the highlighted area similar to FIG. 6A, showing the person's hand inserted in the interior cavity of the glove and showing the gripping zones contacting the person's palm;

FIG. 7A is a front elevation view of a second example of the first embodiment of a glove in accordance with an aspect of the present disclosure, where the glove is an ambidextrous glove having a gripping zone on the palm region adjacent each of the thumb region and little finger region of the glove;

FIG. 7B is a rear elevation view of the glove shown in FIG. 7A;

FIG. 7C is a front elevation view of a third example of the first embodiment, where the glove is an ambidextrous glove having a gripping zone on the palm region adjacent only the thumb region of the glove;

FIG. 7D is a rear elevation view of the glove of FIG. 7C;

FIG. 7E is a front elevation view of a fourth example of the first embodiment, where the glove is an ambidextrous glove having a gripping zone on the palm region adjacent only the little finger region of the glove;

FIG. 7F is a rear elevation view of the glove of FIG. 7E;

FIG. 7G is a front elevation view of a fifth example of the first embodiment, where the glove is an ambidextrous glove having a gripping zone on each of a front and a back of the wrist region only;

FIG. 7H is a rear elevation view of the glove of FIG. 7G;

FIG. 7I is a front elevation view of a sixth example of the first embodiment, where the glove is an ambidextrous glove having a gripping zone on only the front of the wrist region;

FIG. 7J is a rear elevation view of the glove of FIG. 7I;

FIG. 7K is a front elevation view of a sixth example of the first embodiment, where the glove is an ambidextrous glove having a gripping zone on only the second surface of the wrist region;

FIG. 7L is a rear elevation view of the glove of FIG. 7K;

FIG. 8 is a front elevation view of a first example of a second embodiment of a glove in accordance with the present disclosure, wherein the first example is a hand-specific glove shown worn on a left hand of a user, showing discrete gripping zones formed in a palm region and a wrist region of the glove, and showing fingertip regions of reduced circumference;

FIG. 8A is a rear elevation view of the glove of FIG. 8;

FIG. 8B is a front elevation view of a second example of the second embodiment of a glove in accordance with an aspect of the present disclosure, where the glove is an hand-specific glove having a gripping zone on the palm region adjacent each of the thumb region and little finger region of the glove;

FIG. 8C is a rear elevation view of the glove shown in FIG. 8B;

FIG. 8D is a front elevation view of a third example of the second embodiment, where the glove is a hand-specific glove having a gripping zone on the palm region adjacent only the thumb region of the glove;

FIG. 8E is a rear elevation view of the glove of FIG. 8D;

FIG. 8F is a front elevation view of a fourth example of the second embodiment, where the glove is a hand-specific glove having a gripping zone on the palm region adjacent only the little finger region of the glove;

FIG. 8G is a rear elevation view of the glove of FIG. 8F;

FIG. 8H is a front elevation view of a fifth example of the second embodiment, where the glove is a hand-specific glove having a gripping zone on each of a front and a back of the wrist region only;

FIG. 8I is a rear elevation view of the glove of FIG. 8H;

FIG. 8J is a front elevation view of a sixth example of the second embodiment, where the glove is a hand-specific glove having a gripping zone on only the front of the wrist region;

FIG. 8K is a rear elevation view of the glove of FIG. 8J;

FIG. 8L is a front elevation view of a sixth example of the second embodiment, where the glove is a hand-specific glove having a gripping zone on only the back of the wrist region;

FIG. 8M is a rear elevation view of the glove of FIG. 8L;

FIG. 9 is a front elevation view of a first example of a third embodiment of a glove in accordance with the present disclosure, wherein the first example is an ambidextrous glove shown worn on a left hand of a user, showing discrete gripping zones formed in a palm region and a wrist region of the glove;

FIG. 10 is a rear elevation view of the glove illustrated in FIG. 9;

FIG. 11 is a first side elevation view of the glove of FIG. 9;

FIG. 12 is a second side elevation view of the glove of FIG. 9;

FIG. 13A is a front elevation view of a second example of the third embodiment of a glove in accordance with an aspect of the present disclosure, where the glove is an ambidextrous glove having a gripping zone on the palm region adjacent each of the thumb region and little finger region of the glove;

FIG. 13B is a rear elevation view of the glove shown in FIG. 13A;

FIG. 13C is a front elevation view of a third example of the third embodiment, where the glove is an ambidextrous glove having a gripping zone on the palm region adjacent only the thumb region of the glove;

FIG. 13D is a rear elevation view of the glove of FIG. 13C;

FIG. 13E is a front elevation view of a fourth example of the third embodiment, where the glove is an ambidextrous glove having a gripping zone on the palm region adjacent only the little finger region of the glove;

FIG. 13F is a rear elevation view of the glove of FIG. 13E;

FIG. 13G is a front elevation view of a fifth example of the third embodiment, where the glove is an ambidextrous glove having a gripping zone on each of a front and a back of the wrist region only;

FIG. 13H is a rear elevation view of the glove of FIG. 13G;

FIG. 13I is a front elevation view of a sixth example of the third embodiment, where the glove is an ambidextrous glove having a gripping zone on only the front of the wrist region;

FIG. 13J is a rear elevation view of the glove of FIG. 13I;

FIG. 13K is a front elevation view of a seventh example of the third embodiment, where the glove is an ambidextrous glove having a gripping zone on only the second surface of the wrist region;

FIG. 13L is a rear elevation view of the glove of FIG. 13K;

FIG. 14 is a front elevation view of a first example of a fourth embodiment of a glove in accordance with the present disclosure, wherein the first example is a hand-specific glove shown worn on a left hand of a user, and showing discrete gripping zones formed in a palm region and a wrist region of the glove;

FIG. 14A is a rear elevation view of the glove of FIG. 14;

FIG. 14B is a front elevation view of a second example of the fourth embodiment of a glove in accordance with an aspect of the present disclosure, where the glove is an hand-specific glove having a gripping zone on the palm region adjacent each of the thumb region and little finger region of the glove;

FIG. 14C is a rear elevation view of the glove shown in FIG. 14B;

FIG. 14D is a front elevation view of a third example of the fourth embodiment, where the glove is a hand-specific glove having a gripping zone on the palm region adjacent only the thumb region of the glove;

FIG. 14E is a rear elevation view of the glove of FIG. 14D;

FIG. 14F is a front elevation view of a fourth example of the fourth embodiment, where the glove is a hand-specific glove having a gripping zone on the palm region adjacent only the little finger region of the glove;

FIG. 14G is a rear elevation view of the glove of FIG. 14F;

FIG. 14H is a front elevation view of a fifth example of the fourth embodiment, where the glove is a hand-specific glove having a gripping zone on each of a front and a back of the wrist region only;

FIG. 14I is a rear elevation view of the glove of FIG. 14H;

FIG. 14J is a front elevation view of a sixth example of the fourth embodiment, where the glove is a hand-specific glove having a gripping zone on only the front of the wrist region;

FIG. 14K is a rear elevation view of the glove of FIG. 14J;

FIG. 14L is a front elevation view of a sixth example of the fourth embodiment, where the glove is a hand-specific glove having a gripping zone on only the back of the wrist region; and

FIG. 14M is a rear elevation view of the glove of FIG. 14L.

Similar numbers refer to similar parts throughout the drawings.

### DETAILED DESCRIPTION

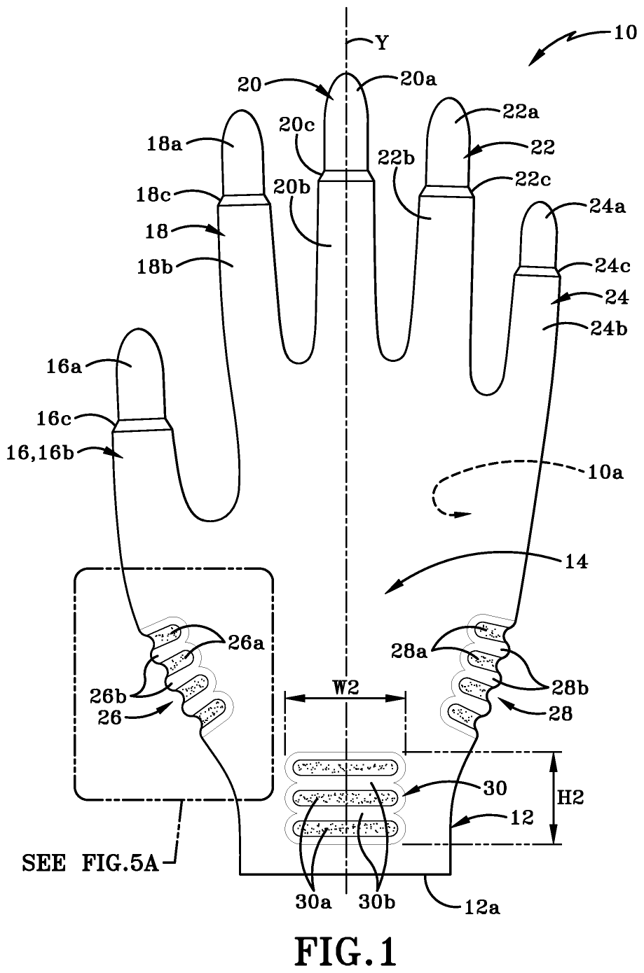

Referring now to FIGS. 1-6B there is shown a first example of a first embodiment of a glove in accordance with the present disclosure, generally indicated at 10. Glove 10 comprises a wrist region 12, a palm region 14, a thumb region 16, an index finger region 18, a middle finger region 20, a ring finger region 22, and a little finger region 24. Glove 10 defines an interior cavity 10a which is configured to receive a hand of a person therein. Thumb region 16, index finger region 18, middle finger region 20, ring finger region 22, and little finger region 24 comprise digit regions which are configured to receive an associated one of a thumb, an index finger, a middle finger, a ring finger, and a little finger of the person's hand, respectively. Palm region 14 is configured to receive the person's palm therein. The palm region is contemplate to include a first surface which will be located adjacent the gripping zones of the person's palms, and a second surface which will be located adjacent the back of the person's hands. The term “palm region” 14 as used herein is contemplated to encompass both of the first surface and the second surface of the glove which will be located adjacent the gripping zone and back of the person's hands, but also the two opposed sides which extend between the first surface and the second surface. In particular, the palm region 14 includes a first side located adjacent an outermost surface of the thumb region 16 and a second side located adjacent an outermost surface of little finger region 24.

Wrist region 12 is that portion of the glove 12 which will cover the person's wrist and, in some embodiments, the forearm of the person when glove 12 is worn. The degree to which the wrist and forearm of the person are covered by wrist region 12 is dependent upon a length of the wrist region 12 as measured from the palm region through to a cuff 12a. A longer length wrist region 12 will cover the wrist and at least part of the forearm of the person wearing the glove. A shorter length wrist region 12 may only just cover the person's wrist. The length of the wrist region 12 will be varied according to the intended final use of glove 12. Consequently, it should be understood that the particular length wrist region 12 shown in the attached figures is exemplary only and shorter or longer wrist regions 12 may be provided on other embodiments of the glove.

The glove 10 disclosed herein is fabricated from any suitable material such as nitrile, nitrile rubber, or natural rubber. The glove material preferably is free of or essentially free of zinc and/or sulfur and/or fluoride, and/or cross-links, and/or accelerators and/or accelerants. Some accelerators/accelerants that the glove's material may be free of or essentially free of may include carbonates or thiurams. The glove material which is free of or essentially free of zinc and/or sulfur and/or fluoride, and/or cross-links, and/or accelerators and/or accelerants will at least comprise a portion of the glove which will contact the user's skin when the glove is worn. The glove material preferably is also formulated to be resistant to a variety of toxic or corrosive compounds or chemicals, such as fentanyl. The composition of the disclosed glove may tend to reduce hypersensitivity or allergic reactions in populations which may be required to frequently wear protective gloves, such as medical practitioners. Additionally, glove 10 may have an exterior surface of one color and an interior surface of another color, particularly a strongly contrasting color. This difference in color between the interior and exterior surfaces of the glove may act as a tear or cut indicator since, if the interior color is visible on the exterior of the glove, the person wearing the glove will realize that the exterior surface has been compromised in some way.

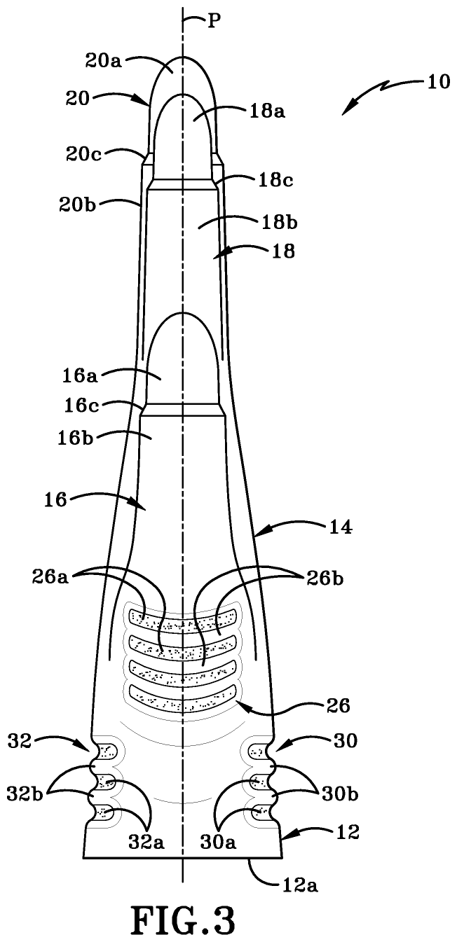

Glove 10 as illustrated in FIGS. 1-6B is an ambidextrous glove which is capable of being worn on either of a left hand or a right hand of the person. In the ambidextrous glove 10 each of the thumb region 16, index finger region 18, middle finger region 20, ring finger region 22, and little finger region 24 are aligned along a common plane “P” (FIG. 3).

As illustrated in each of FIGS. 1-6B, each digit region of glove 10 is comprised of a fingertip region and a remaining portion. The fingertip region originates in a tip of the respective digit region and extends downwardly towards palm region 14 for a distance. The distance is of a length such that the fingertip region will generally be located adjacent to or near where a first knuckle of the person's associated thumb or finger will be when glove 10 is worn. The remaining portion extends between the fingertip region and the palm region 14. The fingertip region of each digit region is of a reduced circumference relative to remaining portion 20b. As illustrated, the remaining portion includes a transition portion which tapers in circumference moving in a direction from the remaining portion of the digit region towards the respective fingertip region thereof.

As illustrated in FIG. 1, thumb region 16 includes a fingertip region 16a, a remaining portion 16b, and a transition portion 16c. Index finger region 18 includes a fingertip region 18a, a remaining portion 18b, and a transition portion 18c. As discussed above, middle finger region 20 includes fingertip region 20a, remaining portion 20b, and transition portion 20c. Ring finger region 22 includes a fingertip region 22a, a remaining portion 22b, and a transition portion 22c. Little finger region 24 includes a fingertip region 24a, a remaining portion 24b, and a transition portion 24c.

FIGS. 1-6B show each of the fingertip regions 16a, 18a, 20a, 22a, and 24a are of a reduced circumference relative to the associated remaining portions 16b, 18b, 20b, 22b, and 24b. Most particularly, the fingertip regions 16a, 18a, 20a, 22a, and 24a are of a “bullet-tip” shape. A bullet-tip shaped fingertip region provided on a glove is described in detail in U.S. Pat. No. 10,602,788 (Hull), which patent is incorporated herein by reference. It should be understood that other configurations of the digit regions which result in a reduced-circumference configuration of the fingertip region may be provided on the digit regions of glove 10 instead of the illustrated bullet-tip configuration.

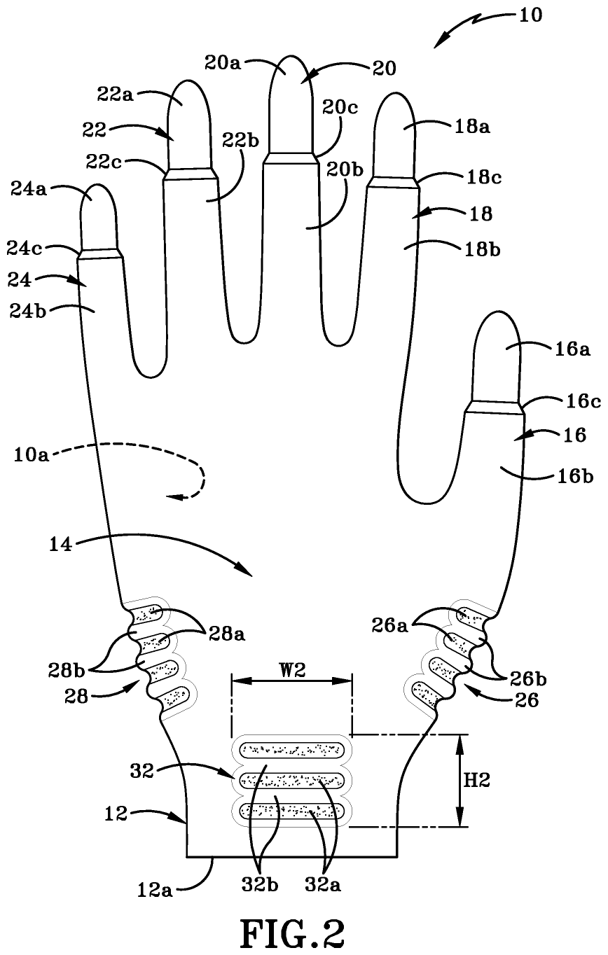

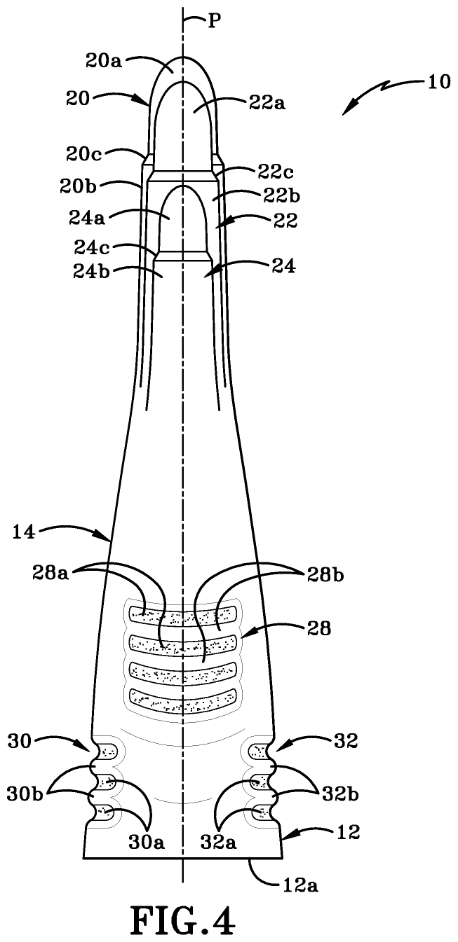

When glove 10 is worn on the left hand as illustrated in FIG. 1, the surface of the glove facing the viewer is considered, for the purposes of this description, to be the “front” surface of the glove 10. The front of the glove is that part of the glove which will contact an object held in a left hand of a person wearing the glove. Because FIG. 1 shows a “front” view, the surfaces of the wrist region 12, palm region 14, and digit regions facing the viewer of FIG. 1 may be considered to be the “front” surface of the respective one of the wrist region 12, palm region 14, and digit regions 16, 18, 20, 22, and 24. FIG. 2 then illustrates a “back” surface of the glove. Consequently, the surface of the wrist region 12, palm region 14, and digit regions facing the viewer in FIG. 2 may be considered to be the “back” surface of each of the wrist region 12, palm region 14, and digit regions 16, 18, 20, 22, and 24, for the purposes of this description. Furthermore, the sides of the wrist region 12, palm region 14, and digit regions 16-24 shown in FIG. 3 may be considered to be the left side or first side of the respective component part of the glove 10 for the purposes of this description. Finally, the sides of the wrist region 12, palm region 14, and digit regions 16-24 shown in FIG. 4 may be considered to be the right side or second side of the respective component part of the glove 10 for the purposes of this description.

It should be noted that the terms “front”, “back”, “left”, and “right” are used for ease of description and particularly describe the specific orientation of the glove 10 illustrated in FIGS. 1 through 4. The terms “front”, “back”, “left”, and “right” should therefore not be narrowly construed to necessarily describe the orientation of the glove 10 during use, particularly since glove 10 is an ambidextrous glove and can be worn on either of the left hand or right hand of a user.

In accordance with an aspect of the present disclosure and as will be discussed in greater detail later herein, glove 10 is provided with at least one gripping zone formed therein. Glove 10 as illustrated in FIGS. 1 and 2 has a first gripping zone 26, a second gripping zone 28, a third gripping zone 30, and a fourth gripping zone 32 (FIG. 2). Glove 10 has an imaginary longitudinal axis “Y” (FIG. 1) which extends from a tip of middle finger region 20 through to cuff 12a. Each of the first gripping zone 26, second gripping zone 28, third gripping zone 30, and fourth gripping zone 32 is oriented generally orthogonal to imaginary longitudinal axis “Y” of glove 10. In particular, first gripping zone 26 is oriented at ninety degrees, i.e., orthogonal to an imaginary longitudinal axis which extends from a tip of thumb region 16 to palm region 14 and second gripping zone 28 is oriented orthogonal to an imaginary longitudinal axis which extends from a tip of little finger region 24 to palm region 14. The imaginary longitudinal axis of thumb region 16 and little finger region 24 are substantially parallel to imaginary longitudinal axis “Y”. Each of the third gripping zone 30 and fourth gripping zone 32 is oriented parallel to an imaginary longitudinal axis extending from a tip of middle finger region 20 to palm region 14. The orthogonal orientations of the stays in the gripping zones helps to ensure maximum friction occurs between the glove material in the gripping zones and the person's skin when the glove is worn.

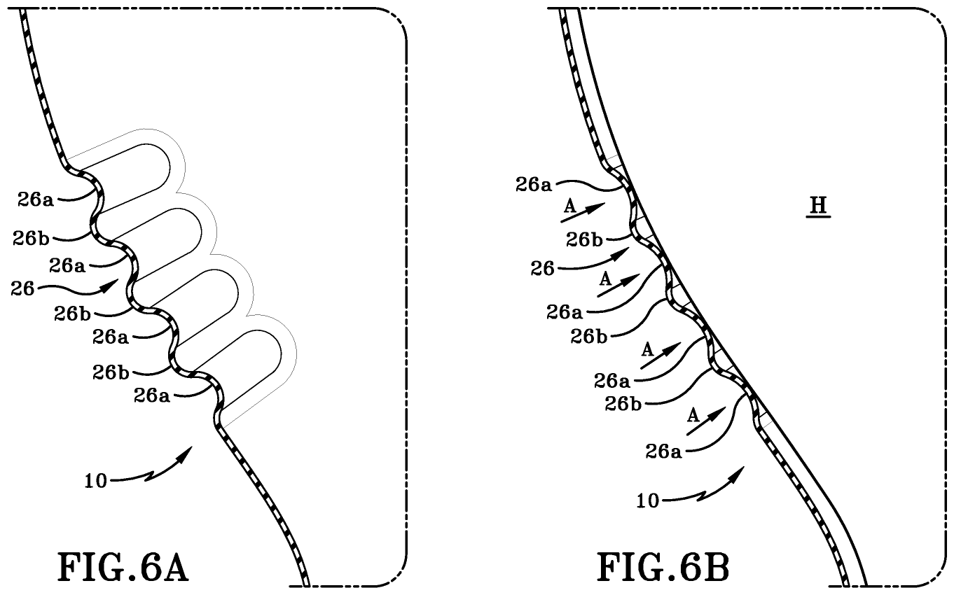

Each of the gripping zones 26, 28, 30, and 32 is of generally a same configuration. Each gripping zone includes a plurality of stays separated by raised regions. Each stay is an arcuate concave indentation formed in a section of the glove material. The concave indentation forms a protrusion which extends into a portion of interior cavity 10a defined by glove 10. This can particularly be seen in FIGS. 6A and 6B. Each of the raised regions is an arcuate convex projection formed in a second of the glove material. Together, the arcuate concave indentations and the arcuate convex projections form a wave structure in the glove material. In one embodiment, the wave structure has the configuration of a sine wave. The wave structure can most readily be seen in FIG. 6A.

FIGS. 1-6B show a configuration of glove 10 in which first gripping zone 26 and second gripping zone 28 are opposed to one other on glove 10 and are laterally spaced and aligned with one another. In other words, an uppermost stay 26a of first gripping zone 26 is laterally spaced from and aligned with an uppermost stay 28a of second gripping zone 28. Similarly, a lowermost stay 26a of first gripping zone 26 is laterally spaced from and aligned with a lowermost stay 28a of second gripping zone 28. (All intermediate stays between the uppermost stay and lowermost stay on the two gripping zones 26, 28 will preferably be aligned with one another.

In one embodiment, the width “W1” and height “H1” of first gripping zone 26 and is substantially the same as the width and height of second gripping zone 28. It will be understood, however, that in other embodiments first gripping zone 26 and second gripping zone 28 may be of different widths and heights relative to one another. Additionally, first gripping zone 26 may be longitudinally offset relative to second gripping zone 28. In other words, the uppermost stay of first gripping zone 26 is not laterally aligned with the uppermost stay of second gripping zone 28.

As shown in FIGS. 1 and 2, first gripping zone 26 comprises a plurality of concave indentations 26a (i.e., stays 26a) which alternate with a plurality of convex projections 26b. Second gripping zone 28 comprises a plurality of concave indentations 28a (i.e., stays 28a) which alternate with a plurality of convex projections 28b. Third gripping zone 30 comprises a plurality of concave indentations 30a (i.e., stays 30a) which alternate with a plurality of convex projections 30b. Fourth gripping zone 32 (FIG. 2) comprises a plurality of concave indentations 32a (i.e., stays 32a) which alternate with a plurality of convex projections 32b.

It should be understood that each of first gripping zone 26, second gripping zone 28, third gripping zone 30, and fourth gripping zone 32 may include one or more concave indentations. Furthermore each gripping zone may include one or more convex projection. As illustrated, first gripping zone 26 comprises four concave indentations 26a and three convex projections 26b but it will be understood that less than four or more than four concave indentations 26a may be formed in first gripping zone 26, and that less than three or more than three convex projections 26b may be formed in first gripping zone 26. Similarly, second gripping zone 28 is illustrated as comprising four concave indentations 28a and three convex projections 28b but it will be understood that less than four or more than four concave indentations 28a may be formed in second gripping zone 28, and that less than three or more than three convex projections 28b may be formed in second gripping zone 28. Third gripping zone 30 is illustrated as comprising three concave indentations 30a and two convex projections 30b but it will be understood that less than three or more than three concave indentations 30a may be formed in third gripping zone 30, and that less than two or more than two convex projections 30b may be formed in third gripping zone 30. Fourth gripping zone 32 is illustrated as comprising three concave indentations 32a and two convex projections 32b but it will be understood that less than three or more than three concave indentations 32a may be formed in fourth gripping zone 32, and that less than two or more than two convex projections 32b may be formed in fourth gripping zone 32.

In accordance with another aspect of the present disclosure, an exterior surface of glove 10 along each of the stays 26a, 28a, 30a, and 32a, is textured. In one embodiment the exterior surface of the glove along the stays of each gripping zone is micro-etched to create the texturing. The micro-etching is accomplished through any process known in the art. In one embodiment, the micro-etching creates a sand pattern texture on the exterior surface of stays 26a, 28a, 30a, and 32a. It will be understood that in other embodiments, any other desired or suitable pattern, texturing or micro-etching may be used. For example, the pattern may be a diamond pattern, a herringbone pattern, or a fish-scale pattern provided on the stays 26a, 28a, 30a, and 32a by micro-etching or by any other suitable process. As illustrated herein, each of the convex projections between stays, such as projections 26b between stays 26a, is free of texturing or micro-etching. The rest of the exterior surface of the glove other than the micro-etched stays of the various gripping zones 26, 28, 30, and 32 is smooth, i.e., free of texturing. The micro-etching strengthens the regions of the glove forming the stays of the gripping zones. While the convex projections 26b have been illustrated herein as being free of texturing or micro-etching, in other embodiments the projections 26b may including texturing or micro-etching while the rest of the glove 10 is free of texturing.

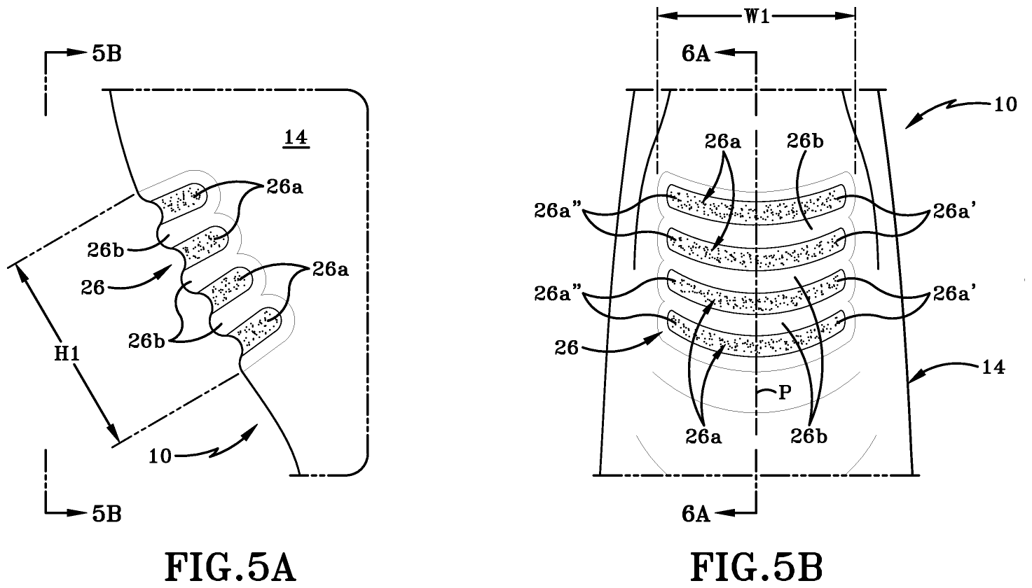

Each gripping zone 26, 28, 30, and 32 is of a desired width and height, where the width is measured from a first end of one of the stays in the gripping zone to the opposed second end of that same stay. The height is measured from an upper end of an uppermost one of the stays to a lower end of a lowermost of the stays. First gripping zone 26 is of a width “W1” (FIG. 5B) measured from the first end 26a′ of stay 26a to a second end 26a″ (FIG. 2) of the same stay 26a. It should be noted that first end 26a′ of stay 26a is on the front of glove 10 and second end 26a″ is on the back of glove 10. First gripping zone 26 is of a height “H1” measured from an upper end of the uppermost stay in first gripping zone 26 to a lower end of the lowermost stay in first gripping zone 26.

Second gripping zone 28, as illustrated, is of a substantially identical width “W1” and height “H1” to first gripping zone 26. The stays 28a of second gripping zone 28, like first gripping zone 26, are formed partially on the front of glove 10, partially on the side of glove 10, and partially on the back of glove 10.

FIG. 1 shows third gripping zone 30 being of a width “W2” and a height “H2” were the width and height of the third gripping zone 30 is measured in a same manner as in the first gripping zone 26 and second gripping zone 28. FIG. 2 shows fourth gripping zone 32 is also of a width “W2” and a height “H2”. The stays 30a of third gripping zone 30 are formed entirely on the front of glove 10 and the stays 32a of fourth gripping zone 32 are formed entirely on the back of glove 10. Third gripping zone 30 and fourth gripping zone 32 are illustrated as being laterally aligned with one another. Third gripping zone 30 and fourth gripping zone 32 are further illustrated to be longitudinally offset from first gripping zone 26 and second gripping zone 28. In particular, third gripping zone 30 and fourth gripping zone 32 are located longitudinally-intermediate lower end 12 of wrist region 12 and the first and second gripping zones 26, 28. In other embodiments, third gripping zone 30 and fourth gripping zone 32 may be longitudinally offset relative to one another instead of the third and fourth gripping zones 30, 32 being laterally aligned with one another.

Each of the first gripping zone 26 and second gripping zone 28 is generally U-shaped in cross-section when viewed looking downwardly towards fingertip region 16a of thumb region 16 and or towards fingertip region 24a of little finger region 24. Each stay 26a of first gripping zone 26 adopts the curvature of the glove 10 which makes the glove suitable for receiving the part of a person's palm and back of the hand below the thumb. Each stay 28a of second gripping zone 28 adopts the curvature of the glove which makes the glove suitable for receiving the part of a person's palm and back of the hand below the little finger. As mentioned above, each of the first gripping zone 26 and second gripping zone 28 is formed partially in the front of palm region 14, partially in the first side (or second side, respectively) of palm region 14, and partially in the back of palm region 14. This arrangement causes first gripping zone 26 to frictionally engage the skin on the front, back and one side of the user's palm, just below the person's thumb. The arrangement further causes second gripping zone 28 to frictionally engage the skin on the front, back, and other side of the user's palm, just below the person's little finger.

Each of the third gripping zone 30 and second gripping zone 32 is generally rectangular or square in shape (as illustrated in FIGS. 1 and 2) and is arrange to grip the skin on the front or back of the person's wrist or forearm, respectively. In particular, the third gripping zone 30 may be arranged to fall generally centrally against the skin of the inside of the person's wrist or forearm while the fourth gripping zone falls generally centrally against the skin of the back of the person's wrist or forearm.

Referring now to FIGS. 1, 6A and 6B in particular, glove 10 is used in the following manner. Wrist region 12 of glove 10 has a cuff end 12a which bounds and defines an opening (not shown) to interior cavity 10a. When a person wishes to don glove 10, he or she will insert their hand into interior cavity 10a through this opening and will slide each of their fingers and thumb into the appropriate one of the digit regions of glove 10 and slide their palm and wrist into the palm region 14 and wrist region 12 of glove 10. FIG. 6B shows that when the person's hand “H” is received in the interior cavity 10a of glove 10, the plurality of concave indentations of the stays 26a will directly contact the skin on the user's hand “H” and will apply a force in the direction indicated by arrow “A” (FIG. 6B), thereby increasing friction between the person's hand “H” and the glove 10. This increased friction will tend to resist creep of the glove relative to the person's hand when the glove is in use. In particular, the increase in friction will tend to prevent wrist region 12 from creeping relative to the person's wrist, and will tend to prevent palm region 14 from creeping relative to the person's palm and back of their hand. Because of the reduction in one or both of wrist region 12 and palm region 14 creep, digit regions 16-24 will tend to remain engaged with the person's thumb and fingers. This helps to ensure that fingertip sensitivity and dexterity are maintained while glove is worn for periods of time that are longer relative to gloves which do not include one or more gripping zones such as first gripping zone 26, second gripping zone 28, third gripping zone 30, and forth gripping zone 32.

It will be understood that the one or more gripping zones 26, 28, 30, and 32 tend to reduce the circumference of the glove 10 in the portions of the glove which include the respective gripping zone, and thereby provide a snug-fit between the person's palm and palm region 14 and the person's wrist and wrist region 12. (The circumference is measured orthogonal to the imaginary longitudinal axis “Y” and passing through one of the stays of the respective gripping zone, such as stay 26a of first gripping zone 26.) This snug-fitting engagement helps to ensure that the person's fingers and thumb have substantially the same tactile sensitivity and dexterity as would be the case if the glove 10 had not been donned.

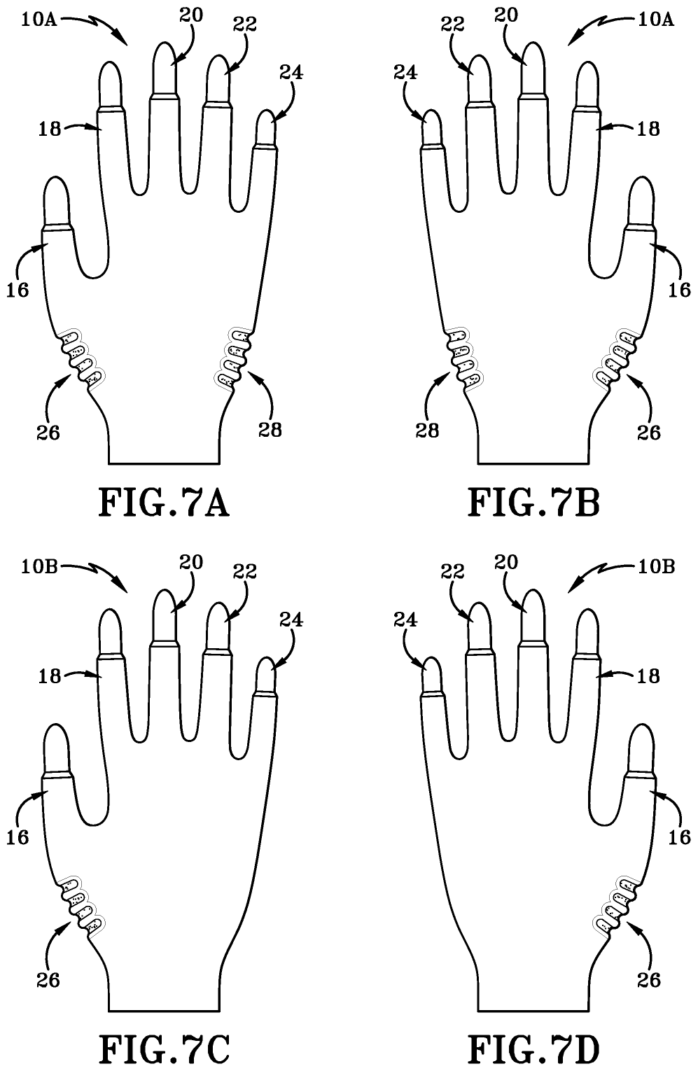

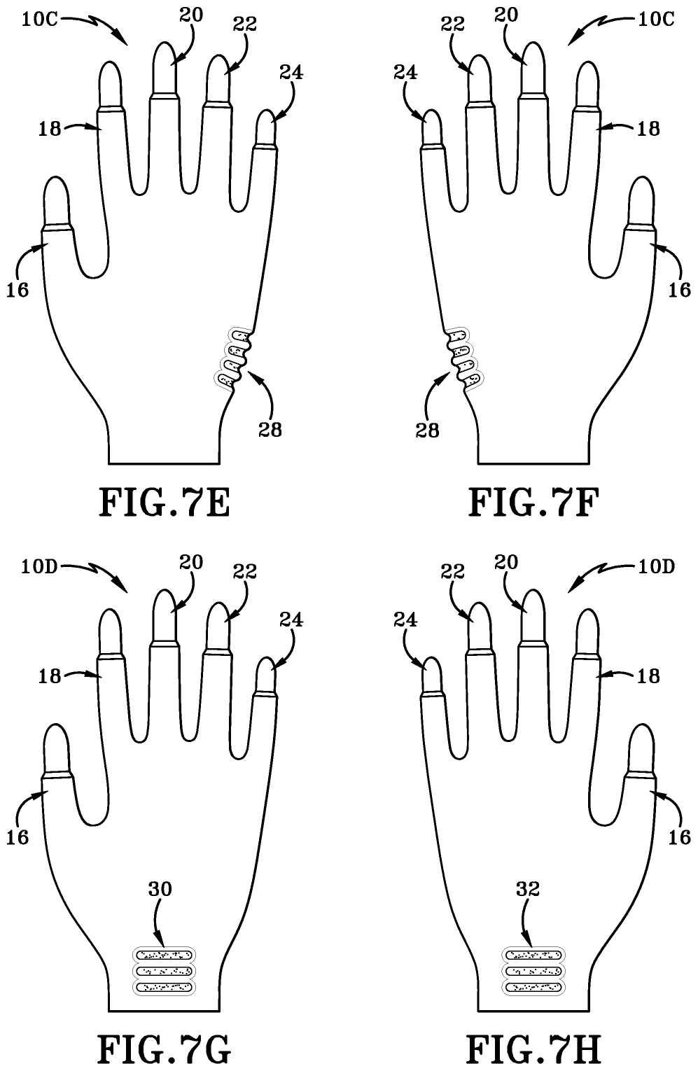

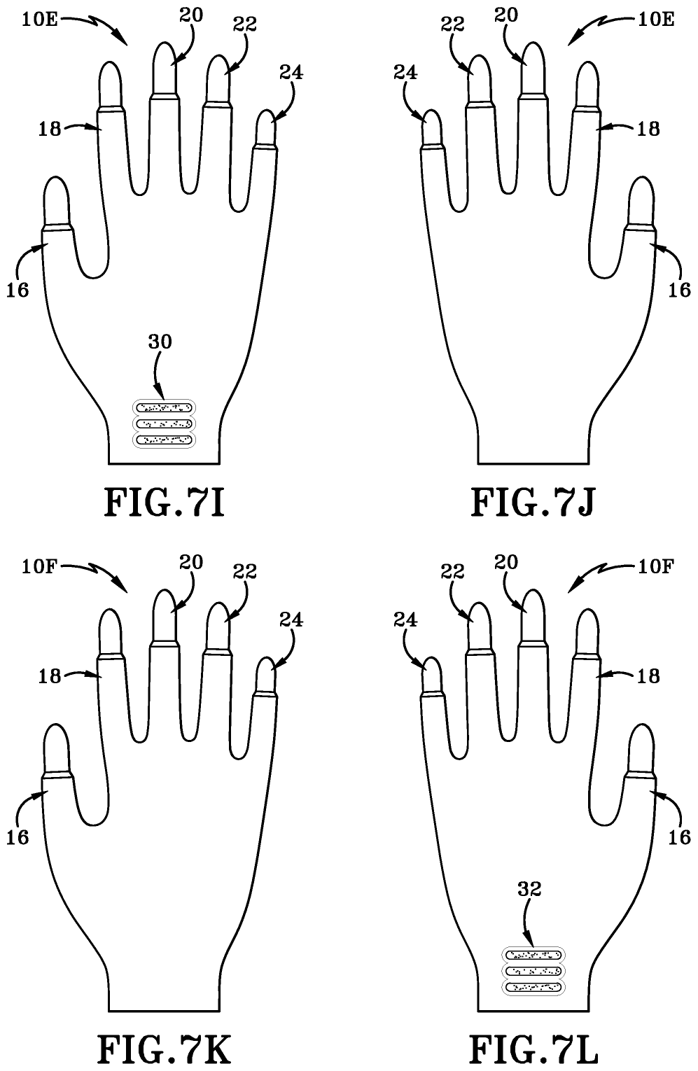

Referring now to FIGS. 7A to 7L there are shown a several additional examples of the first embodiment of glove 10 illustrated in FIGS. 1-6B. FIGS. 7A and 7B show a second example of the first embodiment of the glove, generally indicated as glove 10A. Glove 10A is identical to glove 10 in all aspects except only the first gripping zone 26 and second gripping zone 28 are provided. In other words, the third gripping zone 30 and second gripping zone 32 of glove 10 are omitted in glove 10A. Stated differently, glove 10A only includes a pair of opposed gripping zones in the palm region 14 of glove 10.

FIGS. 7C and 7D show a third example of the first embodiment of the glove, generally indicated as glove 10B. Glove 10B is identical in all aspects to glove 10 except that only the first gripping zone 26 is provided on palm region 14. Each of the second gripping zone 28, third gripping zone 30, and fourth gripping zone 32 of glove 10 are omitted in glove 10B. Stated differently, glove 10B only includes a single gripping zone which is located between the thumb region 16 and a bottom 12a of wrist region 12.

FIGS. 7E and 7F show a fourth example of the first embodiment of the glove, generally indicated as glove 10C. Glove 10C is identical in all aspects to glove 10 except that only the second gripping zone 28 is provided on palm region 14. Each of the first gripping zone 26, third gripping zone 30, and fourth gripping zone 32 of glove 10 are omitted in glove 10C. Stated differently, glove 10C only includes a single gripping zone which is located between the little finger region 24 and a bottom 12a of wrist region 12.

FIGS. 7G and 7H show a fifth example of the first embodiment of the glove, generally indicated as glove 10D. Glove 10D is identical in all aspects to glove 10 except that only the third gripping zone 30 and the fourth gripping zone 32 are provided in wrist region 12. In other words, each of the first gripping zone 26 and second gripping zone 28 is omitted from glove 10D. Stated differently, glove 10D only includes gripping zones which are located in the wrist region 12 and there are no gripping zones in palm region 14.

FIGS. 7I and 7J show a sixth example of the first embodiment of the glove, generally indicated as glove 10E. Glove 10E is identical in all aspects to glove 10 except that only the third gripping zone 30 is provided in wrist region 12. In other words, each of the first gripping zone 26, second gripping zone 28, and fourth gripping zone 32 is omitted from glove 10E. Stated differently, glove 10E only includes a single gripping zone which is located in a front of the wrist region 12 and there are no gripping zones in palm region 14 or in the back of the wrist region 12.

FIGS. 7K and 7L show a seventh example of the first embodiment of the glove, generally indicated as glove 10F. Glove 10F is identical in all aspects to glove 10 except that only the fourth gripping zone 32 is provided in wrist region 12. In other words, each of the first gripping zone 26, second gripping zone 28, and third gripping zone 30 is omitted from glove 10F. Stated differently, glove 10F only includes a single gripping zone which is located in a back of the wrist region 12 and there are no gripping zones in palm region 14 or in the front of the wrist region 12.

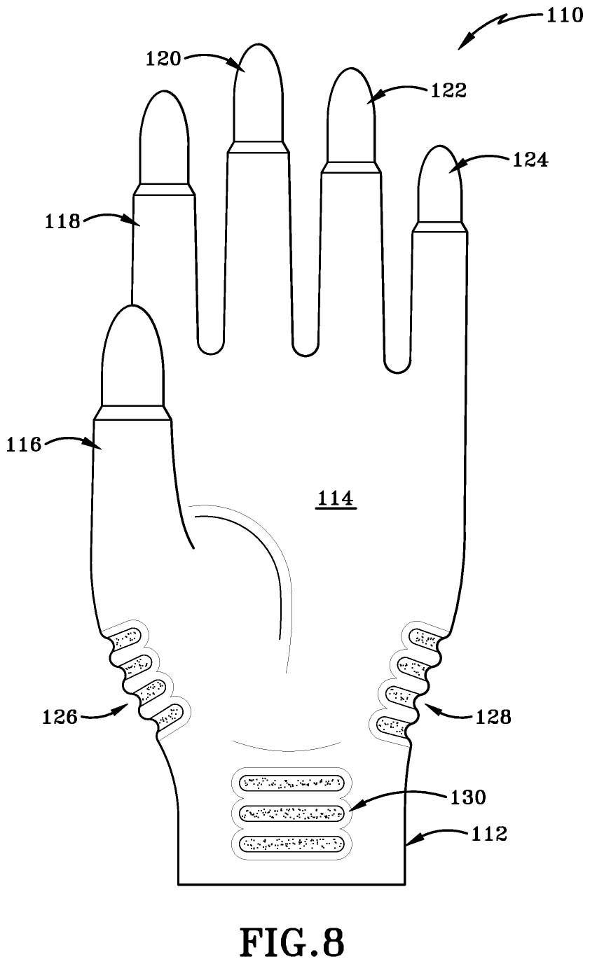

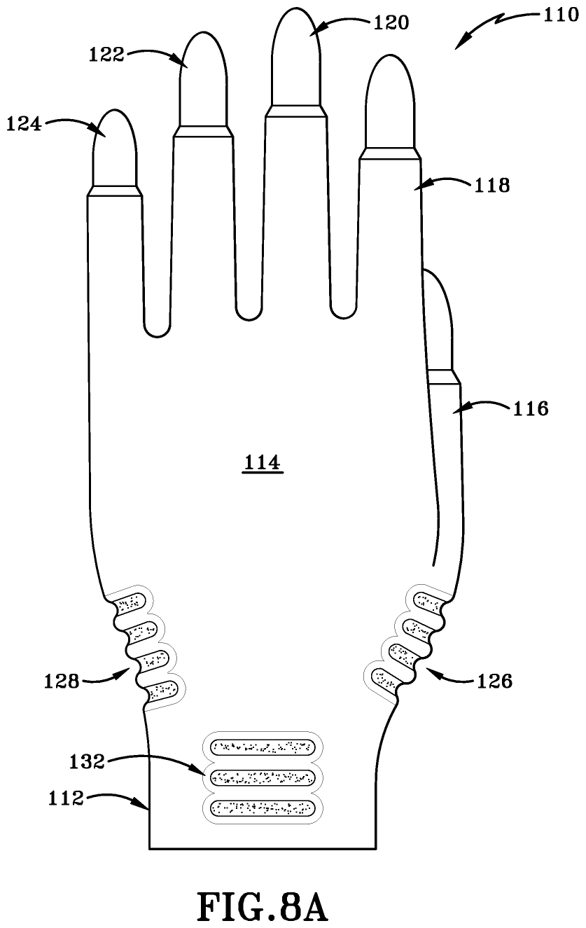

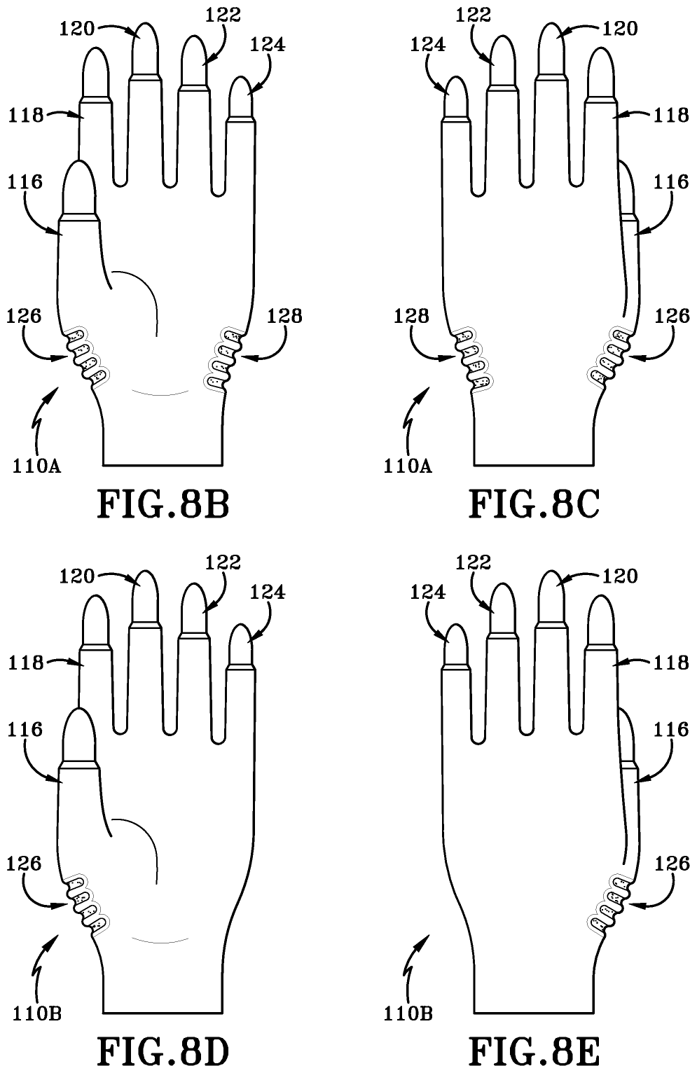

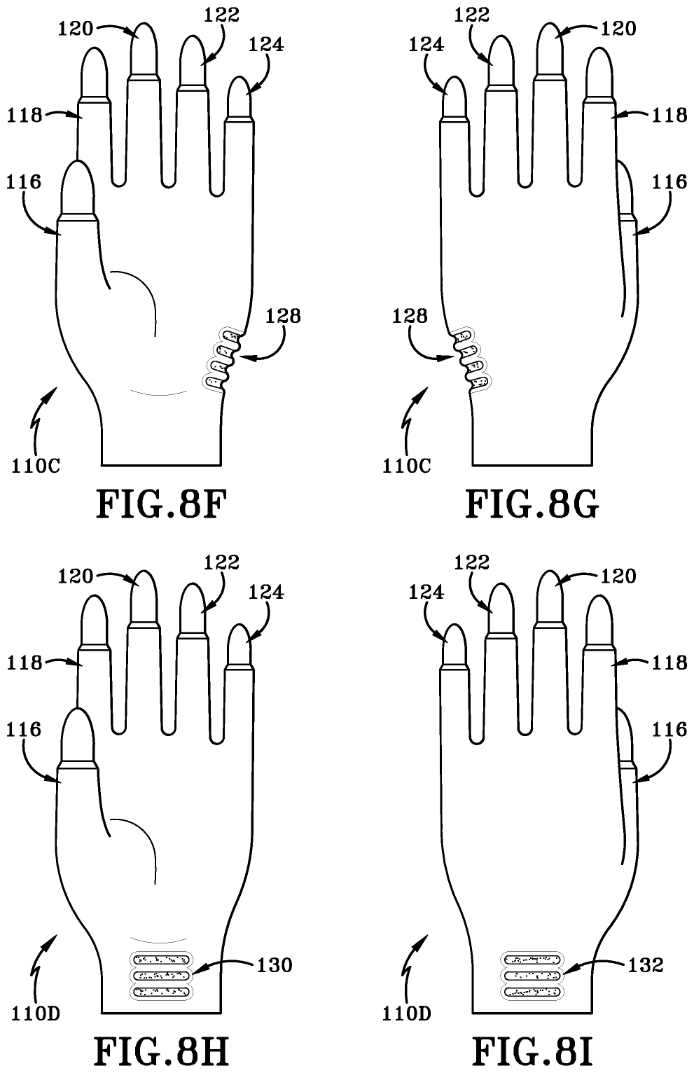

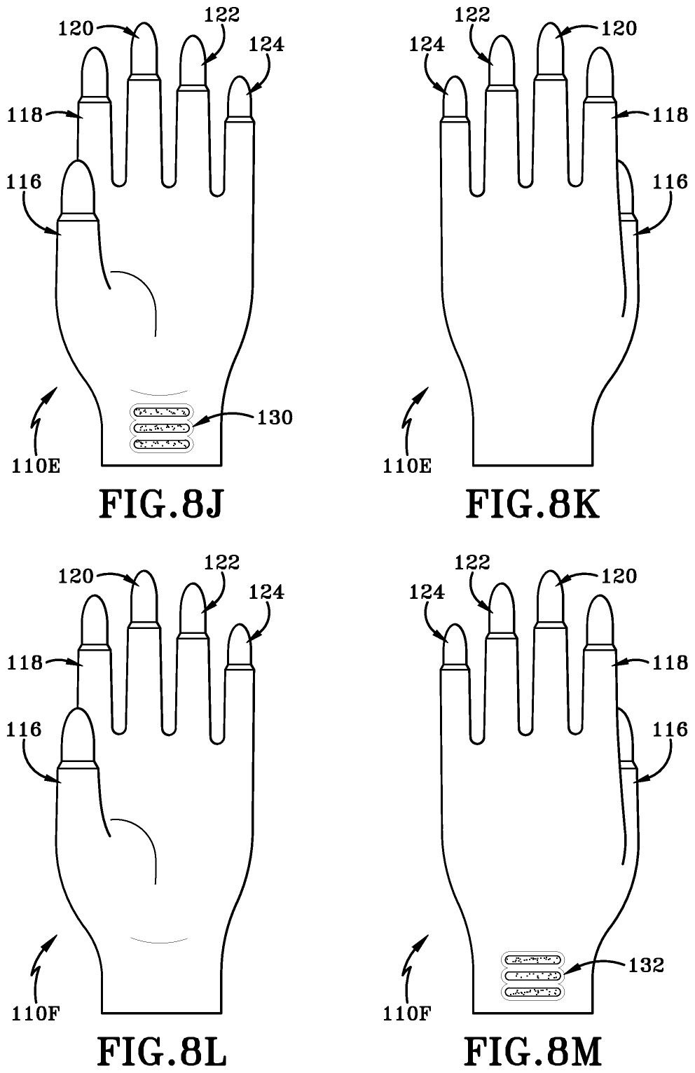

Referring now to FIG. 8 through 8M, there is shown a second embodiment of a glove in accordance with the present disclosure. FIG. 8 shows a first example of the second embodiment of the glove in accordance with the present disclosure, generally indicated at 110. Glove 110 includes a wrist region 112, a palm region 114, and digit regions which extend outwardly from the palm region 114. The digit regions include a thumb region 116, an index finger region 118, a middle finger region 120, a ring finger region 122, and a little finger region 124.

Glove 110 differs from glove 10 in that glove 110 is a hand-specific glove configured to be worn on a left hand of a person, while glove 10 is an ambidextrous glove. The use of glove 110 is substantially identical to that of glove 10 except the glove illustrated in FIGS. 8 and 8A can only be worn on a left hand of a person while glove 10 can be worn on either of the person's left hand or right hand. (It will be understood that a hand-specific glove for the right hand will be a mirror image of the glove 110.)

In the hand-specific glove 110 shown in FIGS. 8 and 8A, the index finger region 118, middle finger region 120, ring finger region 122, and little finger region 124 are aligned along a common plane but the thumb region 116 is in a different plane. Apart from the glove 110 being a hand-specific glove, all of the component parts of the glove 110 are substantially identical in structure and function to glove 10. Other than thumb region 116 being offset from the plane of the other digit regions, thumb region 116 is identical to thumb region 16. Additionally, index finger region 118 is identical to index finger region 18, middle finger region 120 is identical to middle finger region 20, ring finger region 122 is identical to ring finger region 22, and little finger region 124 is identical to little finger region 24.

Although not numbered in FIGS. 8 to 8M, each of the digit regions on glove 110 includes a fingertip region that is identical in structure and function to the associated fingertip region 16a, 18a, 20a, 22a, and 24a of glove 10. Each of the digit regions on glove 110 also includes a remaining portion that is identical in structure and function to the associated remaining portions 16b, 18b, 20b, 22b, and 24b on glove 10. Each of these digit regions of glove 110 also includes a transition portion that is identical in structure and function to the associated transition portion 16c, 18c, 20c, 22c, and 22d of glove 10.

Glove 110 further includes a first gripping zone 126, a second gripping zone 128, a third gripping zone 130, and a fourth gripping zone 132. First gripping zone 126 is substantially identical in structure and function to first gripping zone 26 on glove 10, except first gripping zone 126 is provided on the offset thumb region 116 and therefore may be somewhat wider than the width “W1” of first gripping zone 26. Second gripping zone 128 is substantially identical in structure and function to second gripping zone 28; third gripping zone 130 is substantially identical in structure and function to third gripping zone 30, and fourth gripping zone 132 is substantially identical in structure and function to fourth gripping zone 32. Gripping zones 126, 128, 130, and 132 therefore will not be discussed in any further detail herein.

Referring now to FIGS. 8B to 8M there are shown additional examples of the second embodiment of glove 110 illustrated in FIGS. 8 and 8A. FIGS. 8B and 8C show a second example of the second embodiment of the glove, generally indicated as glove 110A. Glove 110A is identical to glove 110 in all aspects except that third gripping zone 130 and fourth gripping zone 132 are omitted.

FIGS. 8D and 8E show a third example of the second embodiment of the glove, generally indicated as glove 110B. Glove 110B is identical to glove 110 in all aspects except that second gripping zone 128, third gripping zone 130, and fourth gripping zone 132 are omitted.

FIGS. 8F and 8G show a fourth example of the second embodiment of the glove, generally indicated as glove 110C. Glove 110C is identical to glove 110 in all aspects except that first gripping zone 126, third gripping zone 130, and fourth gripping zone 132 are omitted.

FIG. 8H and FIG. 8I show a fifth example of the second embodiment of the glove, generally indicated as glove 110D. Glove 110D is identical to glove 110 in all aspects except that first gripping zone 126 and second gripping zone 128 are omitted.

FIGS. 8J and 8K show a sixth example of the second embodiment of the glove, generally indicated as glove 110E. Glove 110E is identical to glove 110 in all aspects except that first gripping zone 126, second gripping zone 128, and fourth gripping zone 132 are omitted.

FIGS. 8L and 8M show a seventh example of the second embodiment of the glove, generally indicated as glove 110F. Glove 110F is identical to glove 110 in all aspects except that first gripping zone 126, second gripping zone 128, and third gripping zone 130 are omitted.

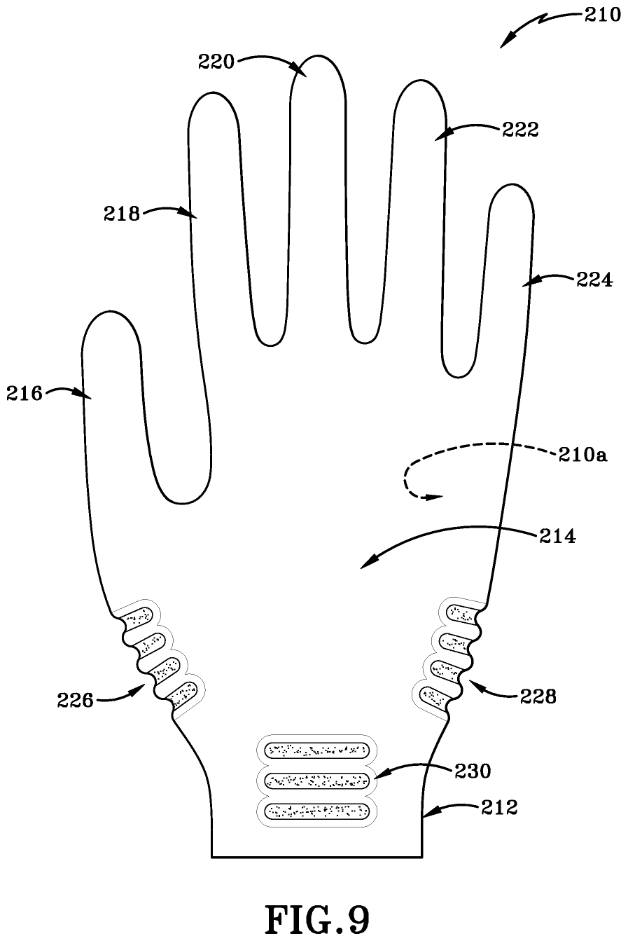

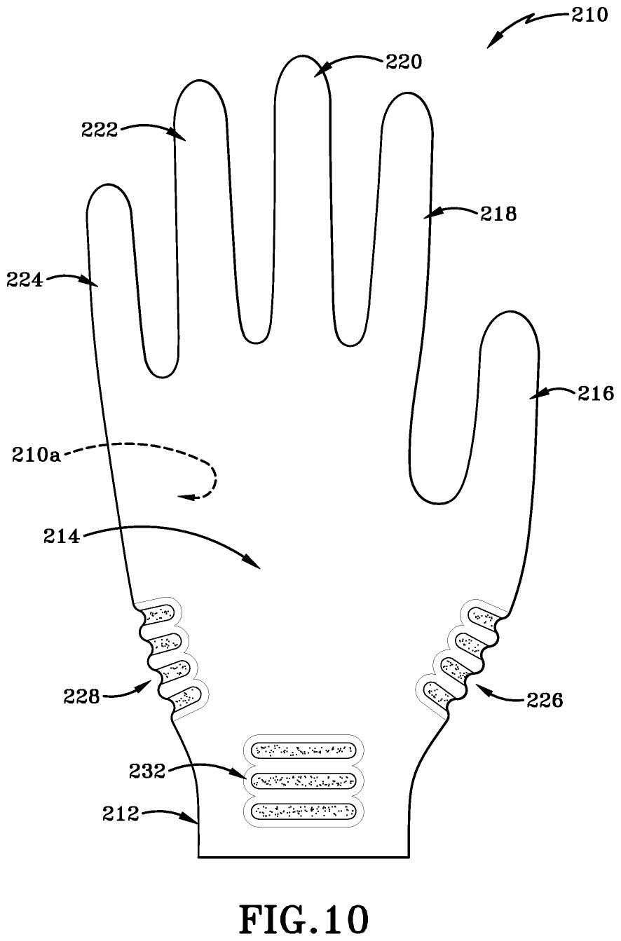

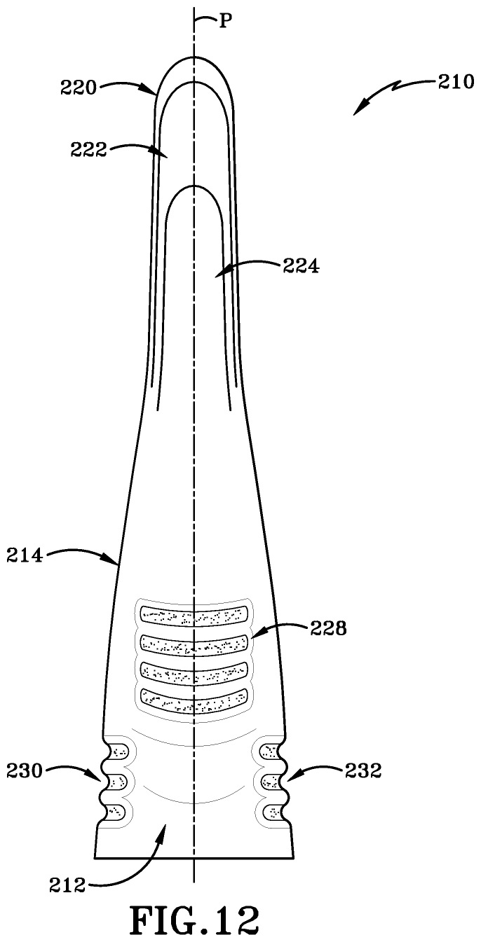

Referring now to FIGS. 9 to 13L there is shown a third embodiment of a glove in accordance with the present disclosure, generally indicated at 210. Glove 210 is substantially identical in structure and function to glove 10 except as shall be described hereafter.

Glove 210 comprises a wrist region 212, a palm region 214, a thumb region 216, an index finger region 218, a middle finger region 220, a ring finger region 222, and a little finger region 224. Glove 210 defines an interior cavity 210a which is configured to receive a hand of a person therein. Thumb region 216, index finger region 218, middle finger region 220, ring finger region 222, and little finger region 224 comprise digit regions which are configured to receive an associated one of a thumb, an index finger, a middle finger, a ring finger, and a little finger of the person's hand, respectively. Palm region 214 is configured to receive a palm and back of the person's hand therein, and wrist region 212 is configured to receive the wrist of the person therein. Depending on a length of wrist region 212, the wrist region 212 may further be configured to receive a portion of the person's forearm therein.

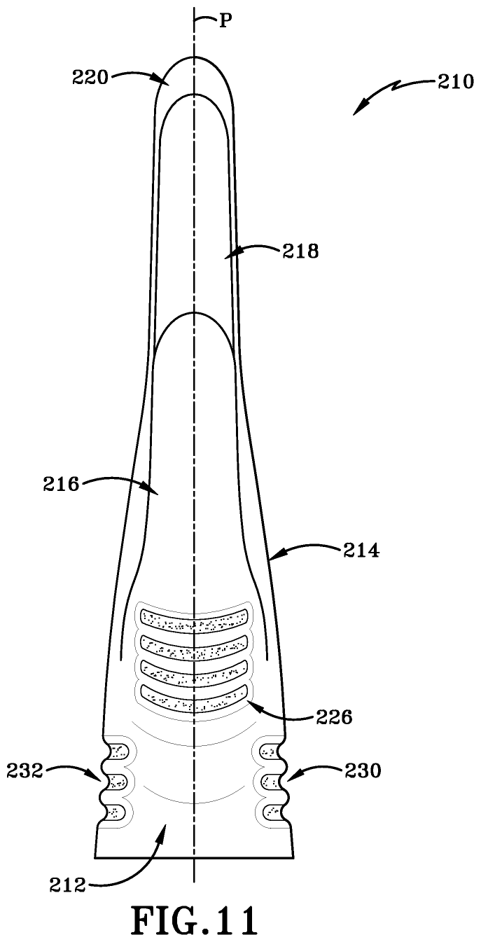

Like glove 10, glove 210 is an ambidextrous glove which is capable of being worn on either of a left hand or a right hand of the person. In the ambidextrous glove 210 each of the thumb region 216, index finger region 218, middle finger region 220, ring finger region 222, and little finger region 224 are aligned along a common plane “P” as shown in FIG. 11.

Glove 210 differs from glove 10 in that all of the digit regions 216, 218, 220, 222, and 224 are of a gradually tapering circumference moving in a direction from palm region 214 to a tip of the respective digit region. In other words, each digit region does not include a reduced-circumference fingertip region which extends from the tip of the digit region to a flaring circumference transition portion, and then to a remaining portion of a larger circumference than the fingertip region. In particular, the digit regions of glove 210 do not have bullet-tip shaped fingertip regions.

All other aspects of glove 210 are substantially identical in structure and function to glove 10. Glove 210 includes a first gripping zone 226, a second gripping zone 228, a third gripping zone 230, and a fourth gripping zone 232 which are respectively identical to first gripping zone 26, second gripping zone 28, third gripping zone 30, and fourth gripping zone 32.

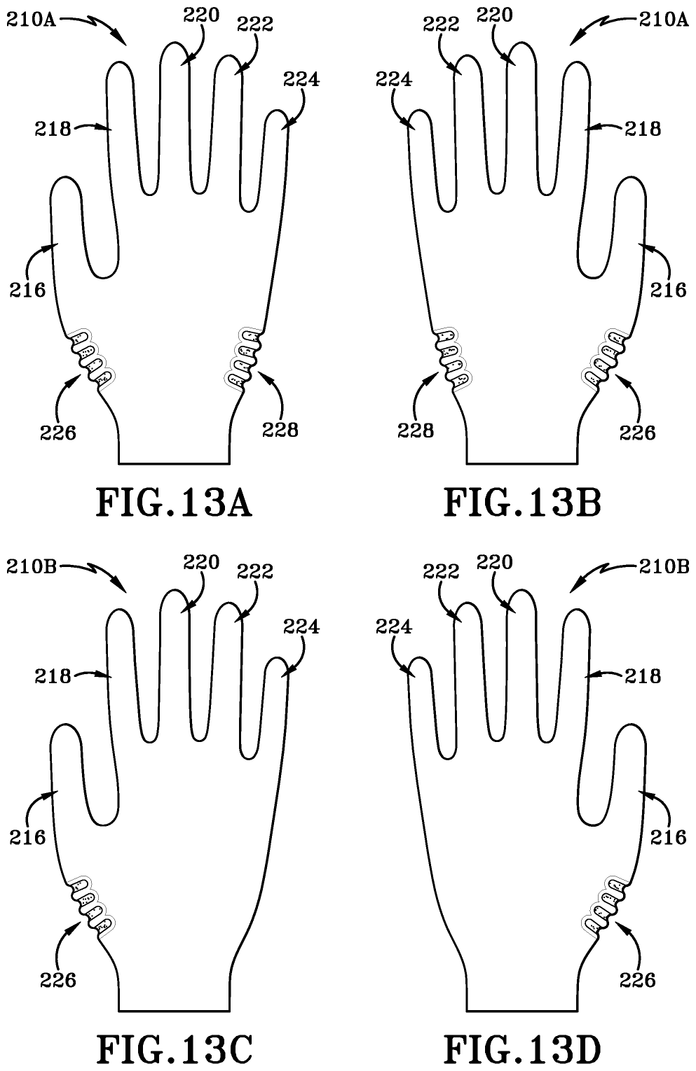

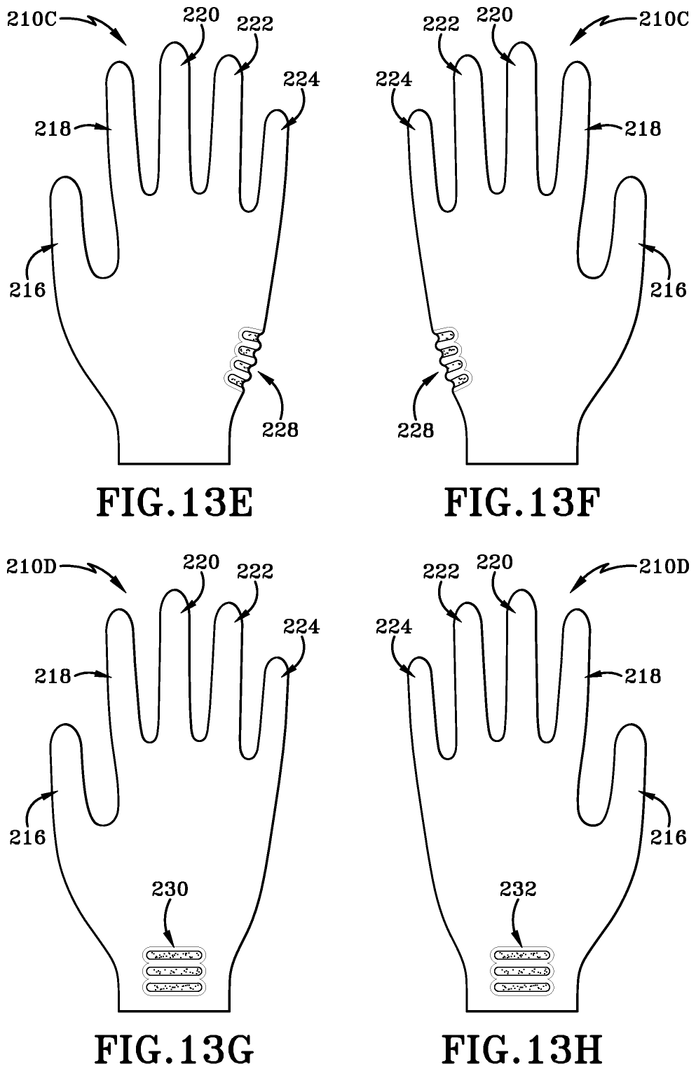

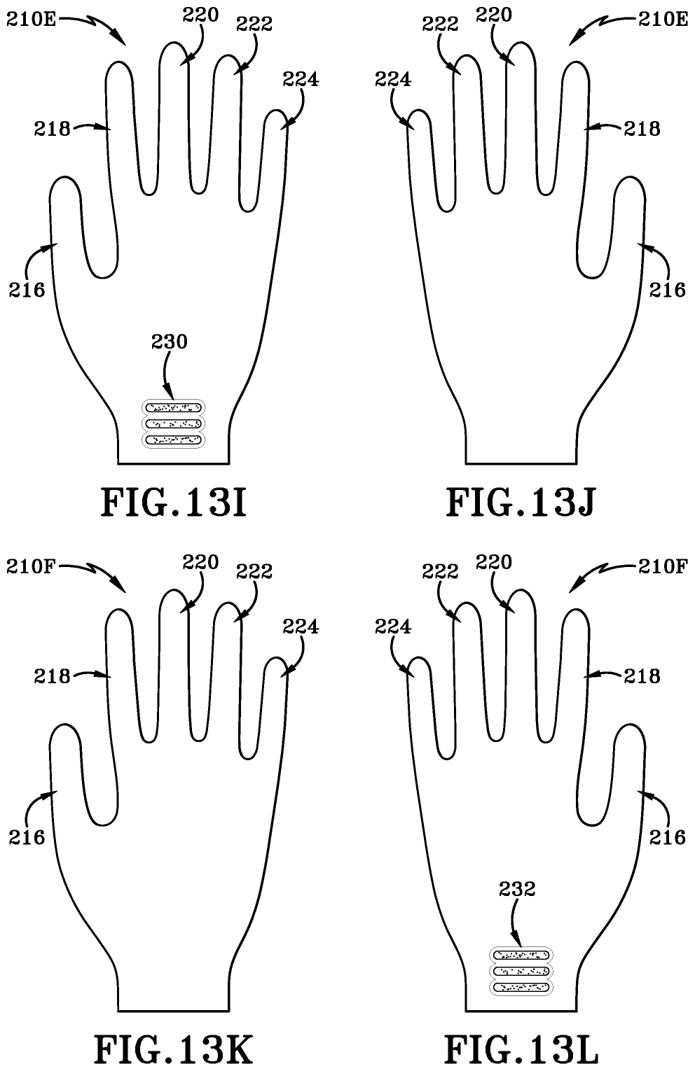

FIGS. 13A through 13L show additional examples of the third embodiment of the glove in accordance with the present disclosure. FIGS. 13A and 13B show a second example of the third embodiment of the glove, generally indicated as glove 210A. Glove 210A is identical to glove 210 in all aspects except that third gripping zone 230 and fourth gripping zone 232 are omitted.

FIGS. 13C and 13D show a third example of the third embodiment of the glove, generally indicated as glove 210B. Glove 210B is identical to glove 210 in all aspects except that second gripping zone 228, third gripping zone 230, and fourth gripping zone 232 are omitted.

FIGS. 13E and 13F show a fourth example of the third embodiment of the glove, generally indicated as glove 210C. Glove 210C is identical to glove 210 in all aspects except that first gripping zone 226, third gripping zone 230, and fourth gripping zone 232 are omitted.

FIG. 13G and FIG. 13H show a fifth example of the third embodiment of the glove, generally indicated as glove 210D. Glove 210D is identical to glove 210 in all aspects except that first gripping zone 226 and second gripping zone 228 are omitted.

FIG. 13I and FIG. 13J show a sixth example of the third embodiment of the glove, generally indicated as glove 210E. Glove 210E is identical to glove 210 in all aspects except that first gripping zone 226, second gripping zone 228, and fourth gripping zone 232 are omitted.

FIGS. 13K and 13L show a seventh example of the third embodiment of the glove, generally indicated as glove 210F. Glove 210F is identical to glove 210 in all aspects except that first gripping zone 226, second gripping zone 228, and third gripping zone 230 are omitted.

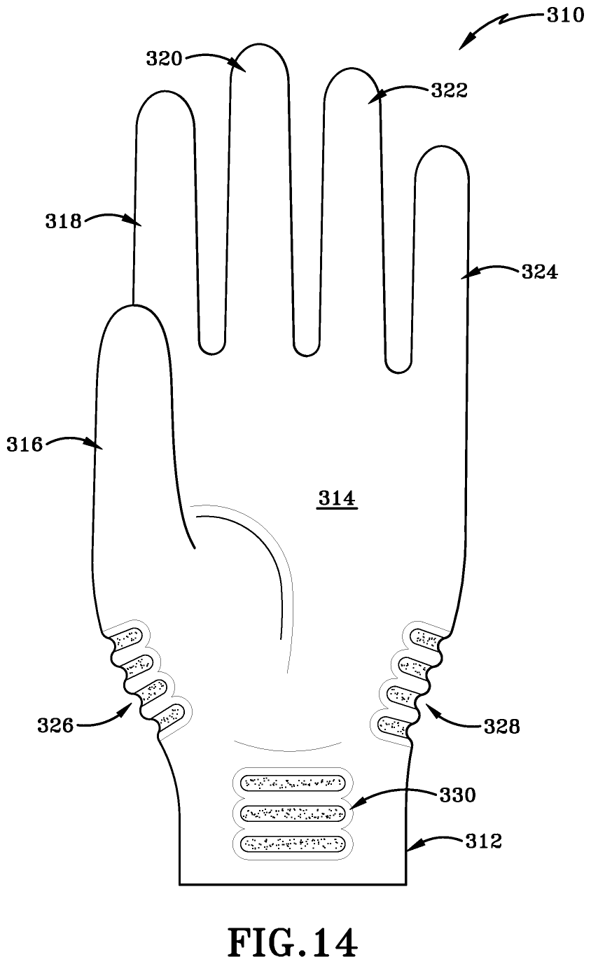

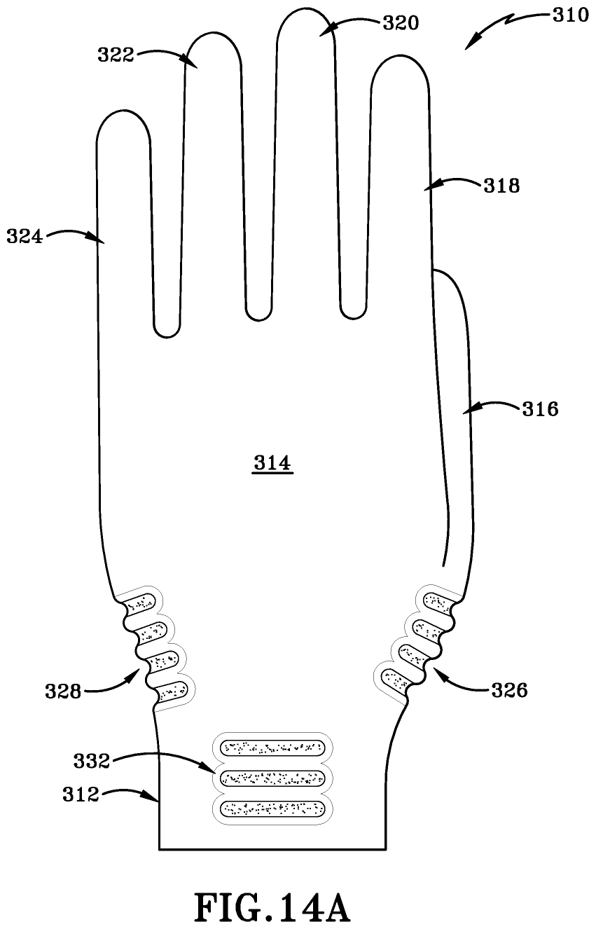

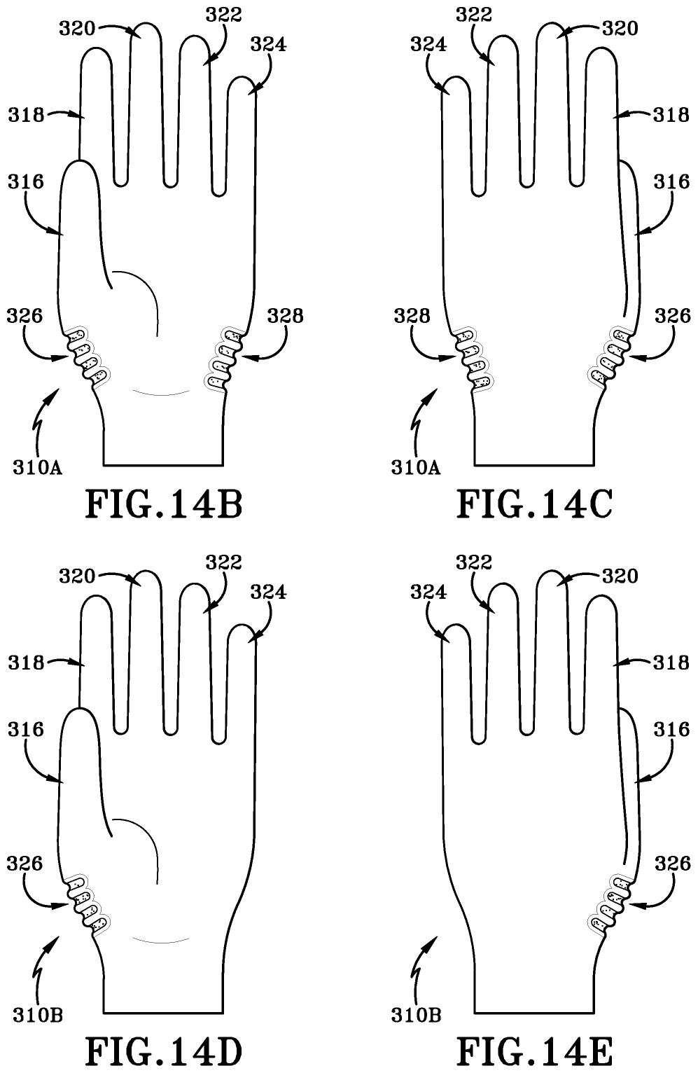

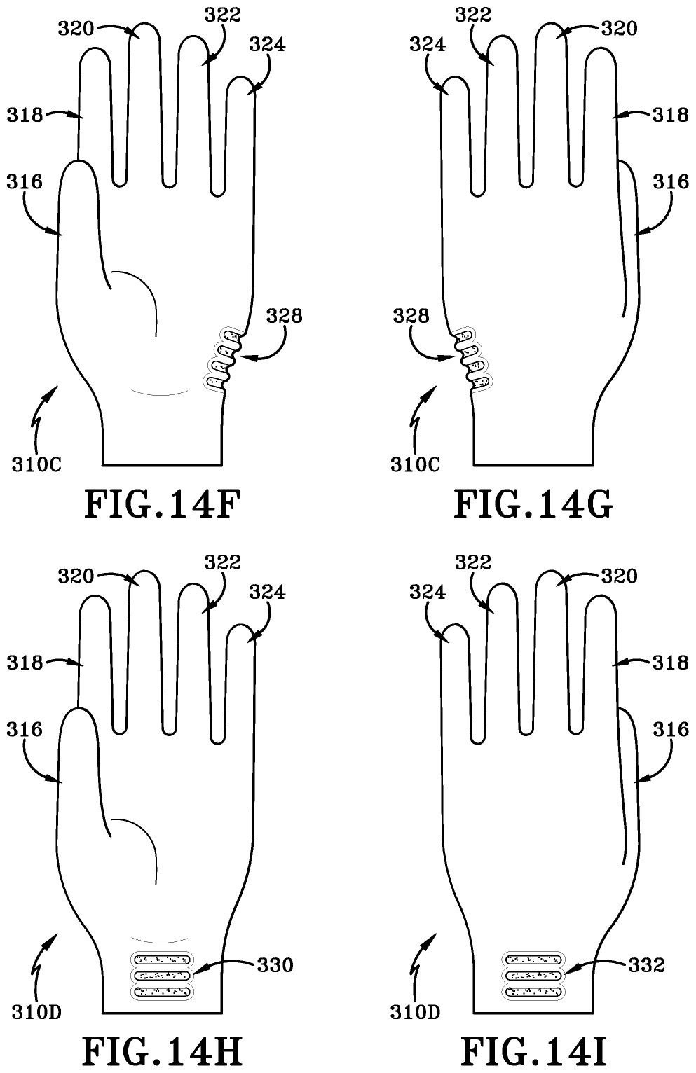

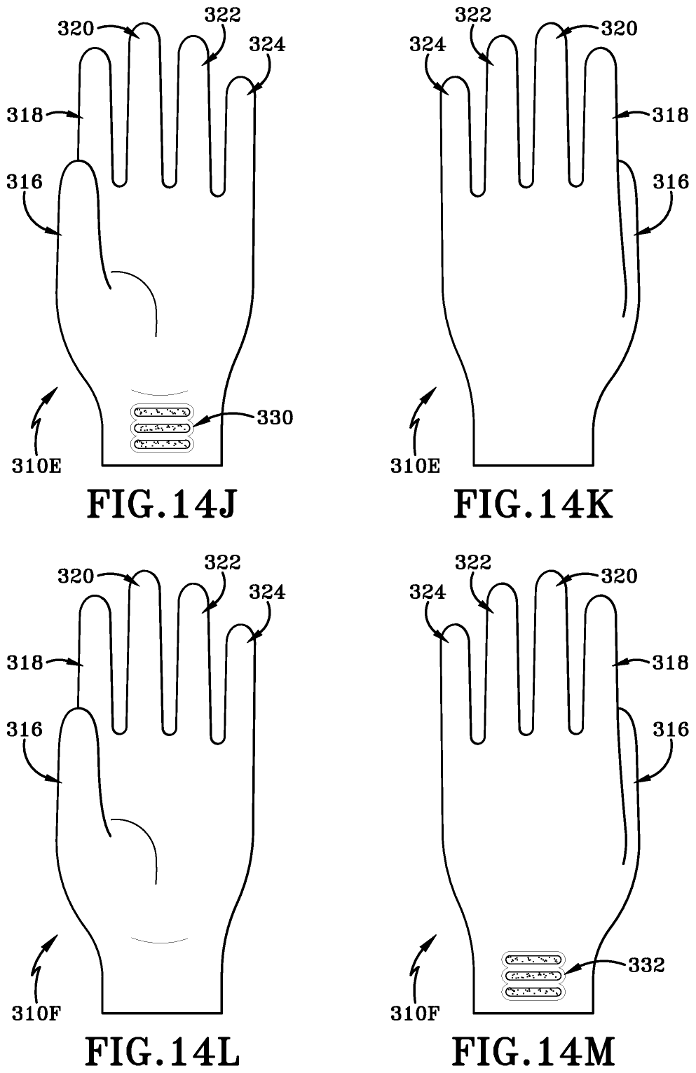

Referring now to FIGS. 14-14M there is shown a fourth embodiment of a glove in accordance with the present disclosure. FIGS. 14 and 14M show a first example of the fourth embodiment of the glove, generally indicated at 310. Glove 310 is substantially identical in structure and function to glove 110 except that instead of each digit region comprising a reduced-circumference fingertip region, a transition portion and a remaining portion as in glove 110, each of the digit regions of glove 310 gradually tapers in circumference from the palm region 314 of the glove to the tip of the associated digit region, similar to glove 210.

Glove 310 is a hand-specific glove (illustrated to be worn on a left had of a person) and comprises a wrist region 312, palm region 314, a thumb region 316, an index finger region 318, a middle finger region 320, a ring finger region 322, and a little finger region 324. (It will be understood that a hand-specific glove for the person's right hand will be a mirror image of the glove 310 shown in FIG. 14) Glove 310 defines an interior cavity 310a which is configured to receive the person's hand. Thumb region 316, index finger region 318, middle finger region 320, ring finger region 322, and little finger region 324 comprise digit regions which are configured to receive an associated one of a thumb, an index finger, a middle finger, a ring finger, and a little finger of the person's hand, respectively. Palm region 314 is configured to receive the person's palm and back of the hand therein, and wrist region 312 is configured to receive the person's wrist therein. As with all other embodiment disclosed herein, the length of wrist region 312 may be varied such that a shorter length wrist region 312 will be configured to only receive a portion of the person's wrist therein, while a longer length wrist region 312 will also receive at least a part of the person's forearm therein.

Glove 310 includes a first gripping zone 326, a second gripping zone 328, a third gripping zone 330, and a fourth gripping zone 332 which are respectively identical to first gripping zone 126, second gripping zone 128, third gripping zone 130, and fourth gripping zone 132 of glove 110.

FIGS. 14B through 14M show additional examples of the fourth embodiment of the glove in accordance with the present disclosure. FIGS. 14B and 14C show a second example of the fourth embodiment of the glove, generally indicated as glove 310A. Glove 310A is identical to glove 310 in all aspects except that third gripping zone 330 and fourth gripping zone 332 are omitted.

FIGS. 14D and 14E show a third example of the fourth embodiment of the glove, generally indicated as glove 310B. Glove 310B is identical to glove 310 in all aspects except that second gripping zone 328, third gripping zone 330, and fourth gripping zone 332 are omitted.

FIGS. 14F and 14G show a fourth example of the fourth embodiment of the glove, generally indicated as glove 310C. Glove 310C is identical to glove 310 in all aspects except that first gripping zone 326, third gripping zone 330, and fourth gripping zone 332 are omitted.

FIG. 14H and FIG. 14I show a fifth example of the fourth embodiment of the glove, generally indicated as glove 310D. Glove 310D is identical to glove 310 in all aspects except that first gripping zone 326 and second gripping zone 328 are omitted.

FIG. 14J and FIG. 14K show a sixth example of the fourth embodiment of the glove, generally indicated as glove 310E. Glove 310E is identical to glove 310 in all aspects except that first gripping zone 326, second gripping zone 328, and fourth gripping zone 332 are omitted.

FIGS. 14L and 14M show a seventh example of the fourth embodiment of the glove, generally indicated as glove 310F. Glove 310F is identical to glove 310 in all aspects except that first gripping zone 326, second gripping zone 328, and third gripping zone 330 are omitted.

The attached figures illustrate that only a single first gripping zone, a single second gripping zone, a single third gripping zone, and a single fourth gripping zone are provided on the various embodiments of the glove disclosed herein. It should be understood, however, that more than one first gripping zone, second gripping zone, third gripping zone, and/or fourth gripping zone may be provided on the glove of the present disclosure.

Various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

The articles “a” and “an,” as used herein in the specification and in the claims, unless clearly indicated to the contrary, should be understood to mean “at least one.” The phrase “and/or,” as used herein in the specification and in the claims (if at all), should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc. As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

While components of the present disclosure are described herein in relation to each other, it is possible for one of the components disclosed herein to include inventive subject matter, if claimed alone or used alone. In keeping with the above example, if the disclosed embodiments teach the features of A and B, then there may be inventive subject matter in the combination of A and B, A alone, or B alone, unless otherwise stated herein.

As used herein in the specification and in the claims, the term “effecting” or a phrase or claim element beginning with the term “effecting” should be understood to mean to cause something to happen or to bring something about. For example, effecting an event to occur may be caused by actions of a first party even though a second party actually performed the event or had the event occur to the second party. Stated otherwise, effecting refers to one party giving another party the tools, objects, or resources to cause an event to occur. Thus, in this example a claim element of “effecting an event to occur” would mean that a first party is giving a second party the tools or resources needed for the second party to perform the event, however the affirmative single action is the responsibility of the first party to provide the tools or resources to cause said event to occur.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Spatially relative terms, such as “under”, “below”, “lower”, “over”, “upper”, “above”, “behind”, “in front of”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal”, “lateral”, “transverse”, “longitudinal”, and the like are used herein for the purpose of explanation only unless specifically indicated otherwise.

Although the terms “first” and “second” may be used herein to describe various features/elements, these features/elements should not be limited by these terms, unless the context indicates otherwise. These terms may be used to distinguish one feature/element from another feature/element. Thus, a first feature/element discussed herein could be termed a second feature/element, and similarly, a second feature/element discussed herein could be termed a first feature/element without departing from the teachings of the present invention.

An embodiment is an implementation or example of the present disclosure. Reference in the specification to “an embodiment,” “one embodiment,” “some embodiments,” “one particular embodiment,” “an exemplary embodiment,” or “other embodiments,” or the like, means that a particular feature, structure, or characteristic described in connection with the embodiments is included in at least some embodiments, but not necessarily all embodiments, of the invention. The various appearances “an embodiment,” “one embodiment,” “some embodiments,” “one particular embodiment,” “an exemplary embodiment,” or “other embodiments,” or the like, are not necessarily all referring to the same embodiments.

If this specification states a component, feature, structure, or characteristic “may”, “might”, or “could” be included, that particular component, feature, structure, or characteristic is not required to be included. If the specification or claim refers to “a” or “an” element, that does not mean there is only one of the element. If the specification or claims refer to “an additional” element, that does not preclude there being more than one of the additional element.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

Additionally, the method of performing the present disclosure may occur in a sequence different than those described herein. Accordingly, no sequence of the method should be read as a limitation unless explicitly stated. It is recognizable that performing some of the steps of the method in a different order could achieve a similar result.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

To the extent that the present disclosure has utilized the term “invention” in various titles or sections of this specification, this term was included as required by the formatting requirements of word document submissions pursuant the guidelines/requirements of the United States Patent and Trademark Office and shall not, in any manner, be considered a disavowal of any subject matter.

In the foregoing description, certain terms have been used for brevity, clearness, and understanding. No unnecessary limitations are to be implied therefrom beyond the requirement of the prior art because such terms are used for descriptive purposes and are intended to be broadly construed.

Moreover, the description and illustration of various embodiments of the disclosure are examples and the disclosure is not limited to the exact details shown or described.

What is claimed is:

1. A glove comprising:
a wrist region;
a palm region extending outwardly from the wrist region;
a plurality of digit regions extending outwardly from the palm region; and
at least one gripping zone provided on at least one of the palm region and the wrist region of the glove.

2. The glove according to claim 1, wherein the at least one gripping zone includes one or more concave indentations formed in a material of the glove, wherein the one or more concave indentations project into an interior cavity of the glove.

3. The glove according to claim 2, wherein the at least one gripping zone further includes one or more convex projections alternating with the one or more concave indentations, wherein the one or more convex projections extend outwardly from an exterior surface of the glove.

4. The glove according to claim 1, wherein the at least one gripping zone is oriented orthogonally to an imaginary longitudinal axis of the glove, wherein the imaginary longitudinal axis extends from a tip of one of the plurality of digit regions to the palm region of the glove.

5. The glove according to claim 1, wherein the plurality of digit regions includes a thumb region, and the at least one gripping zone is provided on the palm region between the thumb region and a bottom end of the wrist region.

6. The glove according to claim 5, wherein the at least one gripping zone is formed partially in a front of the palm region, partially in a first side of the palm region, and partially in a back of the palm region.

7. The glove according to claim 1, wherein the plurality of digit regions includes a little finger region, and the at least one gripping zone is provided on the palm region between the little finger region, and a bottom end of the wrist region.

8. The glove according to claim 7, wherein the at least one gripping zone is formed partially in a front of the palm region, partially in a second side of the palm region, and partially in a back of the palm region.

9. The glove according to claim 1, wherein the plurality of digit regions includes a thumb region and a little finger region, wherein the at least one gripping zone comprises a first gripping zone and a second gripping zone, wherein the first gripping zone is provided on the palm region between the thumb region and a bottom end of the wrist region; and wherein the second gripping zone is provided on the palm region between the little finger region and the bottom end of the wrist region.

10. The glove according to claim 1, wherein the at least one gripping zone comprises one or more gripping zones provided in the wrist region.

11. The glove according to claim 10, wherein the at least one gripping zone further comprises a gripping zone provided in the palm region, and wherein the gripping zone in the palm region is laterally spaced from the gripping zone provided in each of the front and the back of the glove.

12. The glove according to claim 1, further comprising texturing of an exterior surface of at least a portion of the at least one gripping zone.

13. The glove according to claim 12, wherein the texturing is applied through micro-etching.

14. The glove according to claim 1, wherein a digit region of the at least one digit region includes a fingertip region and a remaining portion, wherein the fingertip region originates at a tip of the digit region and extends towards the palm region, wherein the fingertip region terminates in a location which will be adjacent a first knuckle of a person's finger or thumb when the glove is worn; wherein the remaining portion extends from the fingertip region to the palm region; and wherein the fingertip region is of a reduced circumference relative to the remaining portion.

15. The glove according to claim 14, further comprising a transition portion provided on the remaining portion, wherein the transition portion extends outwardly from the fingertip region, and wherein the transition portion tapers in circumference moving in a direction from the palm region towards the fingertip region.

16. The glove according to claim 15, wherein the fingertip region is of a bullet-tip configuration.

17. A method comprising:
inserting a person's hand through an opening defined in a wrist region of a glove;
receiving each finger or thumb of the person's hand into a respective one of a plurality of digit regions of the glove;
receiving a palm and back of the person's hand into a palm region of the glove, wherein the palm region extends outwardly from the plurality of digit regions;
receiving the person's wrist into the wrist region of the glove;
providing at least one gripping zone in at least one of the wrist region and the palm region of the glove; and
frictionally retaining the one of the palm region and the wrist region of the glove in position on the person's palm and back of the hand, or on the person's wrist with the at least one gripping zone.

18. The method according to claim 17, further comprising providing the at least one gripping zone as one or more stays formed in a material of the glove, wherein each of the one or more stays is formed as an arcuate concave indentation which extends into an interior cavity of the glove.

19. The method according to claim 18, further comprises applying texturing on an exterior surface of the at least one gripping zone.

20. The method according to claim 19, wherein the texturing is applied to the exterior surface of the one or more stays via a micro-etching process.
